SEQUENCE LISTING (36 sequences)

SEQ ID NO: 1 - protein, 8 aa, Homo sapiens
GFTFITSA

SEQ ID NO: 2 - protein, 8 aa, Homo sapiens
IAVGSGNT

SEQ ID NO: 3 - protein, 16 aa, Homo sapiens
AAPHCDRTSCHDGFDI

SEQ ID NO: 4 - protein, 123 aa, Homo sapiens
QVQLVESGPEMKKPGTSVKVSCKASGFTFITSAVQWVRQARGQRLEWMGWIAVGSGNTNYAQKFQDRVTINRDMSTSTAYMELSSLRSEDTAVYYCAAPHCDRTSCHDGFDIWGQGTMVTVSS

SEQ ID NO: 5 - protein, 453 aa, Homo sapiens
QVQLVESGPEMKKPGTSVKVSCKASGFTFITSAVQWVRQARGQRLEWMGWIAVGSGNTNYAQKFQDRVTINRDMSTSTAYMELSSLRSEDTAVYYCAAPHCDRTSCHDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 7 aa, Homo sapiens
QSVSSSY

SEQ ID NO: 7 - skipped

SEQ ID NO: 8 - protein, 9 aa, Homo sapiens
QQYGSSPWT

SEQ ID NO: 9 - protein, 108 aa, Homo sapiens
DIQMTQSPGTLSLSPGERATLSCRASQSVSSSYLAWYQQKPGQAPRLLIYGASSRATGIPDRFSGSGSGTDFTLTISRLEPEDFGVYYCQQYGSSPWTFGQGTKVEIK

SEQ ID NO: 10 - protein, 215 aa, Homo sapiens
DIQMTQSPGTLSLSPGERATLSCRASQSVSSSYLAWYQQKPGQAPRLLIYGASSRATGIPDRFSGSGSGTDFTLTISRLEPEDFGVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 7 aa, Homo sapiens
QSVRSSY

SEQ ID NO: 12 - skipped

SEQ ID NO: 13 - protein, 9 aa, Homo sapiens
QQYGSSPWT

SEQ ID NO: 14 - protein, 108 aa, Homo sapiens
DIVMTQSPGTLSLSPGERATLSCRASQSVRSSYLAWYQQKPGQAPRLLIYGASRRGTGIPDRFSGSGSGTDFTLTISRLEPEDFAVYYCQQYGSSPWTFGQGTKVEIK

SEQ ID NO: 15 - protein, 215 aa, Homo sapiens
DIVMTQSPGTLSLSPGERATLSCRASQSVRSSYLAWYQQKPGQAPRLLIYGASRRGTGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 7 aa, Homo sapiens
QSVSSSY

SEQ ID NO: 17 - skipped

SEQ ID NO: 18 - protein, 9 aa, Homo sapiens
QQYGSSPFT

SEQ ID NO: 19 - protein, 108 aa, Homo sapiens
EIVMTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGGGTKVEIK

SEQ ID NO: 20 - protein, 215 aa, Homo sapiens
EIVMTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 7 aa, Homo sapiens
QSVSRNY

SEQ ID NO: 22 - skipped

SEQ ID NO: 23 - protein, 9 aa, Homo sapiens
QQYGSSLFT

SEQ ID NO: 24 - protein, 108 aa, Homo sapiens
EIVLTQSPGTLSLSPGERATLSCRASQSVSRNYLAWYQQKPGQVPRLLIYGASSRATGIPDRFRGSGSGTDFTLTINRLESEDFAVYYCQQYGSSLFTFGPGTKVDIK

SEQ ID NO: 25 - protein, 215 aa, Homo sapiens
EIVLTQSPGTLSLSPGERATLSCRASQSVSRNYLAWYQQKPGQVPRLLIYGASSRATGIPDRFRGSGSGTDFTLTINRLESEDFAVYYCQQYGSSLFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 8 aa, Homo sapiens
GFTFITSA

SEQ ID NO: 28 - protein, 8 aa, Homo sapiens
IAVGSGNT

SEQ ID NO: 29 - protein, 16 aa, Homo sapiens
AAPHCNRTSCHDGFDI

SEQ ID NO: 30 - protein, 123 aa, Homo sapiens
QVQLVESGPEMKKPGTSVKVSCKASGFTFITSAVQWVRQARGQRLEWMGWIAVGSGNTNYAQKFQDRVTINRDMSTSTAYMELSSLRSEDTAVYYCAAPHCNRTSCHDGFDIWGQGTMVTVSS

SEQ ID NO: 31 - protein, 16 aa, Homo sapiens
AAPHCSRTSCHDGFDI

SEQ ID NO: 32 - protein, 123 aa, Homo sapiens
QVQLVESGPEMKKPGTSVKVSCKASGFTFITSAVQWVRQARGQRLEWMGWIAVGSGNTNYAQKFQDRVTINRDMSTSTAYMELSSLRSEDTAVYYCAAPHCSRTSCHDGFDIWGQGTMVTVSS

SEQ ID NO: 33 - protein, 453 aa, Homo sapiens
QVQLVESGPEMKKPGTSVKVSCKASGFTFITSAVQWVRQARGQRLEWMGWIAVGSGNTNYAQKFQDRVTINRDMSTSTAYMELSSLRSEDTAVYYCAAPHCSRTSCHDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 16 aa, Homo sapiens
AAPHCQRTSCHDGFDI

SEQ ID NO: 35 - protein, 123 aa, Homo sapiens
QVQLVESGPEMKKPGTSVKVSCKASGFTFITSAVQWVRQARGQRLEWMGWIAVGSGNTNYAQKFQDRVTINRDMSTSTAYMELSSLRSEDTAVYYCAAPHCQRTSCHDGFDIWGQGTMVTVSS

SEQ ID NO: 36 - protein, 453 aa, Homo sapiens
QVQLVESGPEMKKPGTSVKVSCKASGFTFITSAVQWVRQARGQRLEWMGWIAVGSGNTNYAQKFQDRVTINRDMSTSTAYMELSSLRSEDTAVYYCAAPHCQRTSCHDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0011398 A1
(43) Pub. Date: Jan. 9, 2025

(54) COMPOSTIONS

(71) Applicant: RQ BIOTECHNOLOGY LIMITED, London (GB)

(72) Inventor: Kovilen SAWMYNADEN, London (GB)

(21) Appl. No.: 18/709,419

(22) PCT Filed: Nov. 11, 2022
(86) PCT No.: PCT/EP2022/081676

§ 371 Date: May 10, 2024

(30) Foreign Application Priority Data
Nov. 12, 2021 (GB) 2116386.0
Nov. 12, 2021 (GB) 2116387.8
Nov. 12, 2021 (GB) 2116388.6

(51) Int. Cl. C07K 16/10; A61K 39/00; A61P 31/14; G01N 33/569

(52) U.S. Cl. CPC C07K 16/1003; A61P 31/14; G01N 33/56983; A61K 2039/505; G01N 2333/165

(57) ABSTRACT

The present invention relates to antibodies capable of binding to the spike protein of coronavirus SARS-COV-2, and methods and uses thereof in the prevention, treatment and/or diagnosis of coronavirus infections, and diseases and/or complications associated with coronavirus infections, including COVID-19.

### FIELD OF THE INVENTION

The present invention relates to antigen binding molecules, particularly antibodies, fragments and variants thereof, that bind SARS-COV-2 and the use of said antigen binding molecules in prevention, treatment and/or diagnosis of coronavirus infections including COVID-19.

### BACKGROUND OF THE INVENTION

SARS-COV-2 is a novel beta coronavirus which was first identified as the cause of an outbreak of severe viral respiratory illness in Wuhan, China in January 2020. This disease, subsequently named COVID-19, has been declared a global pandemic.

Several variants of SARS-COV-2 have emerged and are now in circulation. These include Alpha (also known as B. 1.1.7, which was first documented in the United Kingdom in September 2020), Beta (also known as 501Y.V2 or B.1.351, which was first documented in South Africa in May 2020), Gamma (also known as P.1 or 501Y.V2, which was first documented in Brazil in November 2020), Delta (also known as B.1.617.2, which was first documented in India in October 2020) and Omicron (B.1.1.529, which was first documented in multiple countries in November 2021).

Coronaviruses such as SARS-COV-2 have four structural proteins; the nucleocapsid, the envelope, the membrane and the spike(S) proteins. The SARS-COV-2 spike protein forms a homotrimeric structure comprising three spike monomers. Each spike monomer comprises an S1 subunit and an S2 subunit. The S1 subunit further comprises an N-terminal domain (NTD) and a receptor binding domain (RBD). The trimeric spike protein is responsible for engaging target cells and triggering fusion with the target cell membrane via engagement with the angiotensin-converting enzyme 2 (ACE2) cell surface receptor.

Most currently approved SARS-COV-2 vaccines are designed to induce an immune response to the S protein of the original Wuhan variant (hCoV-19/Wuhan/WIV04/2019). However, many of the SARS-COV-2 variants contain mutations in the S protein. The antibodies generated in response to vaccination or previous infection with hCoV-19/Wuhan/WIV04/2019 may therefore be less effective at recognising these and as-yet-unidentified strains, leading to a greater risk of vaccine failure and breakthrough infection, or increased susceptibility to repeat infections in previously infected individuals.

The present invention seeks to provide improved antibodies for preventing, treating and/or diagnosing coronavirus infections, and diseases and/or complications associated with coronavirus infections, including COVID-19 and Long COVID. In particular, the present invention seeks to provide antibodies which have broad activity against multiple known variants of SARS-COV-2 and as-yet-unidentified variants having further mutations in the spike protein.

### SUMMARY OF THE INVENTION

The present invention relates to human monoclonal antibodies (mAbs) that specifically bind to the spike protein of SARS-COV-2 and are effective in neutralising SARS-COV-2.

In some embodiments, the antibodies of the invention specifically bind to an epitope on the S1 subunit of the spike protein. In some embodiments, the antibodies of the invention specifically bind to an epitope on the receptor binding domain (RBD) of the S1 subunit. In alternative embodiments, the antibodies of the invention bind to a quaternary epitope that arises through binding the higher order structures constituting the trimeric spike protein.

Preferably, the antibodies of the invention are broadly effective against multiple variants of SARS-COV-2. For example, the antibodies of the invention may be effective in neutralising some or all of the hCoV-19/Wuhan/WIV04/2019, SARS-COV-2/human/AUS/VIC01/2020 (a hCoV-19/Wuhan/WIV04/2019-related variant, also known as the “Victoria” variant as it was isolated in Australia early in the pandemic), Alpha (B.1.1.7), Beta (B.1.351), Gamma (P1), Delta (B1.617.2), and/or Omicron (B.1.1.529) variants and/or other variants, Variants of Interest (VOIs), and in particular other Variants of Concern (VOCs).

By ‘Variants of Concern’, it is meant variants of SARS-COV-2 which are currently designated as such by the World Health Organisation (WHO) according to their current working definitions or any other relevant criteria. Variants currently designated as ‘Variants of Concern’ are known to the skilled person, and are listed, for example, at https://www.who.int/en/activities/tracking-SARS-COV-2-variants/. Designated ‘Variants of Concern’ as of October 2021 are the Alpha (B.1.1.7), Beta (B.1.351), Gamma (P1) and Delta (B1.617.2) variants.

By ‘Variants of Interest’, it is meant variants of SARS-COV-2 which are currently designated as such by the World Health Organisation (WHO) according to their current working definitions or any other relevant criteria. Variants currently designated as ‘Variants of Interest’ are also known to the skilled person, and are listed, for example, at https://www.who.int/en/activities/tracking-SARS-COV-2-variants/. Designated ‘Variants of Interest’ as of October 2021 are the Lambda (C.37) and Mu (B1.621) variants.

In some embodiments, the antibodies of the invention may be effective in neutralising variants containing particular mutations in the RBD of the spike protein of SARS-COV-2, such as 501Y, 484K, 417N/T, L452R or T478K.

Preferably, the antibodies of the invention result in potent neutralisation of at least two, at least three, at least four, at least five or more than five different variants of SARS-COV-2. In some embodiments, the antibodies of the invention may be effective in neutralising multiple variants and in particular multiple ‘Variants of Concern’, including at least two, at least three, at least four or more than four ‘Variants of Concern’, or at least two, at least three, at least four, or at least all of the hCoV-19/Wuhan/WIV04/2019, SARS-COV-2/human/AUS/VIC01/2020, Alpha, Beta, Gamma and Delta variants.

In some embodiments, the antibodies of the invention may be mixed-chain antibodies generated by swapping the light and heavy chains from two different antibody clones. Preferably, the two different antibody clones are derived from the same public V-genes. For example, the antibodies of the invention may comprise a heavy chain derived from RQCov-01 and a light chain derived from RQCov-02, RQCov-03 or RQCov-04.

Alternatively, the antibodies of the invention may comprise a heavy chain and a light chain derived from the same antibody clone. In some embodiments, the antibodies of the invention may comprise a heavy chain and a light chain derived from RQCov-01.

Preferably, the heavy chain and/or light chain of the antibodies of the invention comprise one or more further modifications, for example to improve the stability or potency of the antibody. In particular, the Fc region of the antibodies of the invention preferably comprises one or more further modifications, for example to improve the stability or potency of the antibody.

In one aspect, the invention provides an antibody capable of binding to the spike protein of coronavirus SARS-COV-2, wherein the antibody comprises a heavy chain variable region comprising a VHCDR1 comprising the amino acid sequence of SEQ ID NO 1, a VHCDR2 comprising the amino acid sequence of SEQ ID NO 2, and a VHCDR3 comprising the amino acid sequence of SEQ ID NO 3, SEQ ID NO: 31 or SEQ ID NO: 34; and/or a light chain variable region comprising:
(a) a VLCDR1 comprising the amino acid sequence of SEQ ID NO: 6, a VLCDR2 comprising the amino acid sequence of SEQ ID NO: 7 and a VLCDR3 comprising the amino acid sequence of SEQ ID NO: 8;
(b) VLCDR1 comprising the amino acid sequence of SEQ ID NO: 11, a VLCDR2 comprising the amino acid sequence of SEQ ID NO: 12 and a VLCDR3 comprising the amino acid sequence of SEQ ID NO: 13;
(c) VLCDR1 comprising the amino acid sequence of SEQ ID NO: 16, a VLCDR2 comprising the amino acid sequence of SEQ ID NO: 17 and a VLCDR3 comprising the amino acid sequence of SEQ ID NO: 18; or
(d) VLCDR1 comprising the amino acid sequence of SEQ ID NO: 21, a VLCDR2 comprising the amino acid sequence of SEQ ID NO: 22 and a VLCDR3 comprising the amino acid sequence of SEQ ID NO: 23.

In some embodiments, the antibody comprises:
(a) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 3 and a light chain variable region comprising a VLCDR1 comprising the amino acid sequence of SEQ ID NO: 6, a VLCDR2 comprising the amino acid sequence of SEQ ID NO: 7, and a VLCDR3 comprising the amino acid sequence of SEQ ID NO: 8;
(b) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 31 and a light chain variable region comprising a VLCDR1 comprising the amino acid sequence of SEQ ID NO: 6, a VLCDR2 comprising the amino acid sequence of SEQ ID NO: 7, and a VLCDR3 comprising the amino acid sequence of SEQ ID NO: 8;
(c) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 34 and a light chain variable region comprising a VLCDR1 comprising the amino acid sequence of SEQ ID NO: 6, a VLCDR2 comprising the amino acid sequence of SEQ ID NO: 7, and a VLCDR3 comprising the amino acid sequence of SEQ ID NO: 8;
(d) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 3 and a light chain variable region comprising a VLCDR1 comprising the amino acid sequence of SEQ ID NO: 11, a VLCDR2 comprising the amino acid sequence of SEQ ID NO: 12, and a VLCDR3 comprising the amino acid sequence of SEQ ID NO: 13;
(e) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 31 and a light chain variable region comprising a VLCDR1 comprising the amino acid sequence of SEQ ID NO: 11, a VLCDR2 comprising the amino acid sequence of SEQ ID NO: 12, and a VLCDR3 comprising the amino acid sequence of SEQ ID NO: 13;
(f) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 34 and a light chain variable region comprising a VLCDR1 comprising the amino acid sequence of SEQ ID NO: 11, a VLCDR2 comprising the amino acid sequence of SEQ ID NO: 12, and a VLCDR3 comprising the amino acid sequence of SEQ ID NO: 13;
(g) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 3 and a light chain variable region comprising a VLCDR1 comprising the amino acid sequence of SEQ ID NO: 16, a VLCDR2 comprising the amino acid sequence of SEQ ID NO: 17, and a VLCDR3 comprising the amino acid sequence of SEQ ID NO: 18;
(h) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 31 and a light chain variable region comprising a VLCDR1 comprising the amino acid sequence of SEQ ID NO: 16, a VLCDR2 comprising the amino acid sequence of SEQ ID NO: 17, and a VLCDR3 comprising the amino acid sequence of SEQ ID NO: 18;
(i) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 34 and a light chain variable region comprising a VLCDR1 comprising the amino acid sequence of SEQ ID NO: 16, a VLCDR2 comprising the amino acid sequence of SEQ ID NO: 17, and a VLCDR3 comprising the amino acid sequence of SEQ ID NO: 18;
(j) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 3 and a light chain variable region comprising a VLCDR1 comprising the amino acid sequence of SEQ ID NO: 21, a VLCDR2 comprising the amino acid sequence of SEQ ID NO: 22, and a VLCDR3 comprising the amino acid sequence of SEQ ID NO: 23;
(k) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 31 and a light chain variable region comprising a VLCDR1 comprising the amino acid sequence of SEQ ID NO: 21, a VLCDR2 comprising the amino acid sequence of SEQ ID NO: 22, and a VLCDR3 comprising the amino acid sequence of SEQ ID NO: 23; or
(l) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 34 and a light chain variable region comprising a VLCDR1 comprising the amino acid sequence of SEQ ID NO: 21, a VLCDR2 comprising the amino acid sequence of SEQ ID NO: 22, and a VLCDR3 comprising the amino acid sequence of SEQ ID NO: 23.

In one embodiment, the invention provides an antibody capable of binding to the spike protein of coronavirus SARS-COV-2, wherein the antibody comprises: (a) a heavy chain variable region comprising a VHCDR1 comprising the amino acid sequence of SEQ ID NO 1, a VHCDR2 comprising the amino acid sequence of SEQ ID NO 2, and a VHCDR3 comprising the amino acid sequence of SEQ ID NO 3, SEQ ID NO: 31 or SEQ ID NO: 34 and (b) a light chain variable region comprising a VLCDR1 comprising the amino acid sequence of SEQ ID NO: 6, a VLCDR2 comprising the amino acid sequence of SEQ ID NO: 7, and a VLCDR3 comprising the amino acid sequence of SEQ ID NO: 8.

In one embodiment, the invention provides an antibody capable of binding to the spike protein of coronavirus SARS-COV-2, wherein the antibody comprises: (a) a heavy chain variable region comprising a VHCDR1 comprising the amino acid sequence of SEQ ID NO 1, a VHCDR2 comprising the amino acid sequence of SEQ ID NO 2, and a VHCDR3 comprising the amino acid sequence of SEQ ID NO 3, SEQ ID NO: 31 or SEQ ID NO: 34 and (b) a light chain variable region comprising a VLCDR1 comprising the amino acid sequence of SEQ ID NO: 11, a VLCDR2 comprising the amino acid sequence of SEQ ID NO: 12, and a VLCDR3 comprising the amino acid sequence of SEQ ID NO: 13.

In one embodiment, the invention provides an antibody capable of binding to the spike protein of coronavirus SARS-COV-2, wherein the antibody comprises: (a) a heavy chain variable region comprising a VHCDR1 comprising the amino acid sequence of SEQ ID NO 1, a VHCDR2 comprising the amino acid sequence of SEQ ID NO 2, and a VHCDR3 comprising the amino acid sequence of SEQ ID NO 3, SEQ ID NO: 31 or SEQ ID NO: 34 and (b) a light chain variable region comprising a VLCDR1 comprising the amino acid sequence of SEQ ID NO: 16, a VLCDR2 comprising the amino acid sequence of SEQ ID NO: 17, and a VLCDR3 comprising the amino acid sequence of SEQ ID NO: 18.

In one embodiment, the invention provides an antibody capable of binding to the spike protein of coronavirus SARS-COV-2, wherein the antibody comprises: (a) a heavy chain variable region comprising a VHCDR1 comprising the amino acid sequence of SEQ ID NO 1, a VHCDR2 comprising the amino acid sequence of SEQ ID NO 2, and a VHCDR3 comprising the amino acid sequence of SEQ ID NO 3, SEQ ID NO: 31 or SEQ ID NO: 34; and (b) a light chain variable region comprising a VLCDR1 comprising the amino acid sequence of SEQ ID NO: 21, a VLCDR2 comprising the amino acid sequence of SEQ ID NO: 22, and a VLCDR3 comprising the amino acid sequence of SEQ ID NO: 23.

In some embodiments, the antibody comprises: (a) a heavy chain variable domain having at least 80% sequence identity to SEQ ID NO: 4, SEQ ID NO: 32 or SEQ ID NO: 35 and/or a light chain variable domain having at least 80% sequence identity to SEQ ID NO: 9, 14, 19, or 24.

In some embodiments, the antibody comprises: (a) a heavy chain variable domain having at least 85%, at least 90%, at least 95%, at least 96%, at least 97%, at least 98%, at least 99%, or greater than 99% sequence identity to SEQ ID NO: 4, SEQ ID NO: 32 or SEQ ID NO: 35 and/or (b) a light chain variable domain at least 85%, at least 90%, at least 95%, at least 96%, at least 97%, at least 98%, at least 99%, or greater than 99% sequence identity to SEQ ID NO: 9, 14, 19, or 24.

In some embodiments, the antibody comprises: (a) a heavy chain variable domain having the sequence of SEQ ID NO: 4, SEQ ID NO: 32 or SEQ ID NO: 35 and (b) a light chain variable domain having the sequence of SEQ ID NO: 9, 14, 19, or 24.

For example, the antibody may comprise a heavy chain comprising a heavy chain variable domain having the sequence of SEQ ID NO: 4, SEQ ID NO: 32 or SEQ ID NO: 35 and a light chain comprising a light chain variable domain having the sequence of SEQ ID NO: 9, 14, 19, or 24.

In some embodiments, the antibody comprises:
(a) a heavy chain variable domain having at least 80% sequence identity to SEQ ID NO: 4 and a light chain variable domain having at least 80% sequence identity to SEQ ID NO: 9;
(b) a heavy chain variable domain having at least 80% sequence identity to SEQ ID NO: 32 and a light chain variable domain having at least 80% sequence identity to SEQ ID NO: 9; (c) a heavy chain variable domain having at least 80% sequence identity to SEQ ID NO: 35 and a light chain variable domain having at least 80% sequence identity to SEQ ID NO: 9;
(d) a heavy chain variable domain having at least 80% sequence identity to SEQ ID NO: 4 and a light chain variable domain having at least 80% sequence identity to SEQ ID NO: 14;
(e) a heavy chain variable domain having at least 80% sequence identity to SEQ ID NO: 32 and a light chain variable domain having at least 80% sequence identity to SEQ ID NO: 14;
(f) a heavy chain variable domain having at least 80% sequence identity to SEQ ID NO: 35 and a light chain variable domain having at least 80% sequence identity to SEQ ID NO: 14;
(g) a heavy chain variable domain having at least 80% sequence identity to SEQ ID NO: 4 and a light chain variable domain having at least 80% sequence identity to SEQ ID NO: 19;
(h) a heavy chain variable domain having at least 80% sequence identity to SEQ ID NO: 32 and a light chain variable domain having at least 80% sequence identity to SEQ ID NO: 19;
(i) a heavy chain variable domain having at least 80% sequence identity to SEQ ID NO: 35 and a light chain variable domain having at least 80% sequence identity to SEQ ID NO: 19;
(j) a heavy chain variable domain having at least 80% sequence identity to SEQ ID NO: 4 and a light chain variable domain having at least 80% sequence identity to SEQ ID NO: 24;
(k) a heavy chain variable domain having at least 80% sequence identity to SEQ ID NO: 32 and a light chain variable domain having at least 80% sequence identity to SEQ ID NO: 24; or
(l) a heavy chain variable domain having at least 80% sequence identity to SEQ ID NO: 35 and a light chain variable domain having at least 80% sequence identity to SEQ ID NO: 24.

In some embodiments, the antibody comprises: (a) a heavy chain variable domain having at least 80%, at least 85%, at least 90%, at least 95%, at least 96%, at least 97%, at least 98%, at least 99%, or greater than 99% sequence identity to SEQ ID NO: 4, SEQ ID NO: 32 or SEQ ID NO: 35 and (b) a light chain variable domain having at least 80%, at least 85%, at least 90%, at least 95%, at least 96%, at least 97%, at least 98%, at least 99%, or greater than 99% sequence identity to SEQ ID NO: 9.

In some embodiments, the antibody comprises: (a) a heavy chain variable domain having at least 80%, at least 85%, at least 90%, at least 95%, at least 96%, at least 97%, at least 98%, at least 99%, or greater than 99% sequence identity to SEQ ID NO: 4, SEQ ID NO: 32 or SEQ ID NO: 35 and (b) a light chain variable domain having at least 80%, at least 85%, at least 90%, at least 95%, at least 96%, at least 97%, at least 98%, at least 99%, or greater than 99% sequence identity to SEQ ID NO: 14.

In some embodiments, the antibody comprises: (a) a heavy chain variable domain having at least 80%, at least 85%, at least 90%, at least 95%, at least 96%, at least 97%, at least 98%, at least 99%, or greater than 99% sequence identity to SEQ ID NO: 4, SEQ ID NO: 32 or SEQ ID NO: 35, and (b) a light chain variable domain having at least 80%, at least 85%, at least 90%, at least 95%, at least 96%, at least 97%, at least 98%, at least 99%, or greater than 99% sequence identity to SEQ ID NO: 19.

In some embodiments, the antibody comprises: (a) a heavy chain variable domain having at least 80%, at least 85%, at least 90%, at least 95%, at least 96%, at least 97%, at least 98%, at least 99%, or greater than 99% sequence identity to SEQ ID NO: 4, SEQ ID NO: 32 or SEQ ID NO: 35 and (b) a light chain variable domain having at least 80%, at least 85%, at least 90%, at least 95%, at least 96%, at least 97%, at least 98%, at least 99%, or greater than 99% sequence identity to SEQ ID NO: 24.

In a further aspect, the invention provides a bispecific antibody comprising (a) a first heavy chain comprising a heavy chain variable domain having the sequence of SEQ ID NO: 4, SEQ ID NO: 32 or SEQ ID NO: 35; (b) a first light chain comprising a light chain variable domain having the sequence of SEQ ID NO: 9, 14, 19, or 24; (c) a second heavy chain; and (d) a second light chain.

Preferably, the first heavy chain associates with the first light chain to form a first binding domain specifically binding to an epitope on the spike protein of SARS-COV-2 and the second heavy chain associates with the second light chain to form a second binding domain.

Preferably, the second heavy chain is derived from a different antibody clone to the first heavy chain.

In some embodiments, the first light chain has the same sequence as the second light chain. For example, both the first and second light chains are light chains comprising a light chain variable domain having the sequence of SEQ ID NO: 9, 14, 19, or 24.

In alternative embodiments, the first light chain does not have the same sequence as the second light chain. For example, the first light chain comprises a light chain variable domain having a sequence selected from SEQ ID NO: 9, 14, 19, or 24, and the second light chain comprises a light chain variable domain having a different sequence selected from SEQ ID NO: 9, 14, 19, or 24.

In some embodiments, the second light chain is derived from a different antibody clone to the first light chain. For example, the second light chain may be derived from the same antibody clone as the second heavy chain.

Thus, in some embodiments, the first light chain comprises a light chain variable domain having a sequence selected from SEQ ID NO: 9, 14, 19, or 24, and the second light chain comprises a light chain variable domain that does not have a sequence selected from SEQ ID NO: 9, 14, 19, or 24.

In some embodiments, the bispecific antibody of the invention comprises: (a) a first heavy chain comprising a heavy chain variable domain having the sequence of SEQ ID NO: 4, SEQ ID NO: 32 or SEQ ID NO: 35; (b) a second heavy chain derived from a different antibody clone to the first heavy chain; and (c) a first and second light chain comprising a light chain variable domain having the sequence of SEQ ID NO: 9.

In some embodiments, the bispecific antibody of the invention comprises: (a) a first heavy chain comprising a heavy chain variable domain having the sequence of SEQ ID NO: 4, SEQ ID NO: 32 or SEQ ID NO: 35; (b) a second heavy chain derived from a different antibody clone to the first heavy chain; and (c) a first and second light chain comprising a light chain variable domain having the sequence of SEQ ID NO: 14.

In some embodiments, the bispecific antibody of the invention comprises: (a) a first heavy chain comprising a heavy chain variable domain having the sequence of SEQ ID NO: 4, SEQ ID NO: 32 or SEQ ID NO: 35; (b) a second heavy chain derived from a different antibody clone to the first heavy chain; and (c) a first and second light chain comprising a light chain variable domain having the sequence of SEQ ID NO: 19.

In some embodiments, the bispecific antibody of the invention comprises: (a) a first heavy chain comprising a heavy chain variable domain having the sequence of SEQ ID NO: 4, SEQ ID NO: 32 or SEQ ID NO: 35; (b) a second heavy chain derived from a different antibody clone to the first heavy chain; and (c) a first and second light chain comprising a light chain variable domain having the sequence of SEQ ID NO: 24.

In some embodiments, the second binding domain may bind to the same epitope on the spike protein of SARS-COV-2 as the first binding domain. In other embodiments, the second binding domain may bind a different epitope on the spike protein of SARS-COV-2. In certain embodiments, the second binding domain may bind to a secondary or quaternary epitope on the trimeric spike of SARS-COV-2. In other embodiments, the second binding domain may bind to a protein other than the spike protein of SARS-COV-2. The different protein may be another SARS-COV-2 protein or a non-SARS-COV2 protein.

In some embodiments, the antibody comprises: (a) a heavy chain having at least 80% sequence identity to SEQ ID NO: 5, SEQ ID NO: 33 or SEQ ID NO: 36 and/or (b) a light chain having at least 80% sequence identity to SEQ ID NO: 10, 15, 20 or 25.

In some embodiments, the antibody comprises: (a) a heavy chain having at least 85%, at least 90%, at least 95%, at least 96%, at least 97%, at least 98%, at least 99%, or greater than 99% sequence identity to SEQ ID NO: 5, SEQ ID NO: 33 or SEQ ID NO: 36 and/or (b) a light chain having at least 85%, at least 90%, at least 95%, at least 96%, at least 97%, at least 98%, at least 99%, or greater than 99% sequence identity to SEQ ID NO: 10, 15, 20 or 25.

In some embodiments, the antibody comprises: (a) a heavy chain having at least 85%, at least 90%, at least 95%, at least 96%, at least 97%, at least 98%, at least 99%, or greater than 99% sequence identity to SEQ ID NO: 5, SEQ ID NO: 33 or SEQ ID NO: 36 and (b) a light chain having at least 85%, at least 90%, at least 95%, at least 96%, at least 97%, at least 98%, at least 99%, or greater than 99% sequence identity to SEQ ID NO: 10.

In some embodiments, the antibody comprises: (a) a heavy chain having at least 85%, at least 90%, at least 95%, at least 96%, at least 97%, at least 98%, at least 99%, or greater than 99% sequence identity to SEQ ID NO: 5, SEQ ID NO: 33 or SEQ ID NO: 36 and (b) a light chain having at least 85%, at least 90%, at least 95%, at least 96%, at least 97%, at least 98%, at least 99%, or greater than 99% sequence identity to SEQ ID NO: 15.

In some embodiments, the antibody comprises: (a) a heavy chain having at least 85%, at least 90%, at least 95%, at least 96%, at least 97%, at least 98%, at least 99%, or greater than 99% sequence identity to SEQ ID NO: 5, SEQ ID NO: 33 or SEQ ID NO: 36 and (b) a light chain having at least 85%, at least 90%, at least 95%, at least 96%, at least 97%, at least 98%, at least 99%, or greater than 99% sequence identity to SEQ ID NO: 20.

In some embodiments, the antibody comprises: (a) a heavy chain having at least 85%, at least 90%, at least 95%, at least 96%, at least 97%, at least 98%, at least 99%, or greater than 99% sequence identity to SEQ ID NO: 5, SEQ ID NO: 33 or SEQ ID NO: 36 and (b) a light chain having at least 85%, at least 90%, at least 95%, at least 96%, at least 97%, at least 98%, at least 99%, or greater than 99% sequence identity to SEQ ID NO: 25.

In some embodiments, the antibody comprises: (a) a heavy chain having the sequence of SEQ ID NO: 5, SEQ ID NO: 33 or SEQ ID NO: 36, optionally comprising one or more amino acid substitutions in the heavy chain constant regions and (b) a light chain having the sequence of SEQ ID NO: 10, 15, 20 or 25, optionally comprising one or more amino acid substitutions in the light chain constant region.

In some embodiments, the antibody comprises (a) a heavy chain having the sequence of SEQ ID NO: 5, SEQ ID NO: 33 or SEQ ID NO: 36, optionally comprising one or more amino acid substitutions in the heavy chain constant regions and (b) a light chain derived from a different antibody clone to the heavy chain.

In some embodiments, the antibody comprises (a) a light chain having the sequence of SEQ ID NO: 10 15, 20 or 25, optionally comprising one or more amino acid substitutions and in the light chain constant region (b) a heavy chain derived from a different antibody clone to the light chain.

In a further aspect, the invention provides a bispecific antibody comprising (a) a first heavy chain having the sequence of SEQ ID NO: 5, SEQ ID NO: 33 or SEQ ID NO: 36, optionally comprising one or more amino acid substitutions in the heavy chain constant region; (b) a first light chain having the sequence of SEQ ID NO: 10, 15, 20 or 25, optionally comprising one or more amino acid substitutions in the light chain constant region, (c) a second heavy chain; and (d) a second light chain.

Preferably, the first heavy chain associates with the first light chain to form a first binding domain specifically binding to an epitope on the spike protein of SARS-COV-2 and the second heavy chain associates with the second light chain to form a second binding domain.

In some embodiments, the antibody comprises (a) a first heavy chain having the sequence of SEQ ID NO: 5, SEQ ID NO: 33 or SEQ ID NO: 36, optionally comprising one or more amino acid substitutions in the heavy chain constant region; (b) a first light chain having the sequence of SEQ ID NO: 10, 15, 20 or 25, optionally comprising one or more amino acid substitutions in the light chain constant region (c) a second heavy chain derived from a different antibody clone to the first heavy chain; and (d) a second light chain.

In some embodiments, the first light chain has the same sequence as the second light chain. For example, both the first and second light chains have the sequence of SEQ ID NO: 10, 15, 20 or 25, optionally comprising one or more amino acid substitutions in the light chain constant region.

In alternative embodiments, the first light chain has a sequence selected from SEQ ID NO: 10, 15, 20 or 25, optionally comprising one or more amino acid substitutions in the light chain constant region, and the second light chain has a different sequence selected from SEQ ID NO: 10, 15, 20 or 25, optionally comprising one or more amino acid substitutions in the light chain constant region.

In some embodiments, the second light chain is derived from a different antibody clone to the first light chain. For example, the second light chain may be derived from the same antibody clone as the second heavy chain.

In some embodiments, the bispecific antibody comprises: (a) a first heavy chain having the sequence of SEQ ID NO: 5, SEQ ID NO: 33 or SEQ ID NO: 36, optionally comprising one or more amino acid substitutions in the heavy chain constant region; (b) a second heavy chain derived from a different antibody clone to the first heavy chain; and (c) a first and second light chain having the sequence of SEQ ID NO: 10, optionally comprising one or more amino acid substitutions in the light chain constant region.

In some embodiments, the bispecific antibody comprises: (a) a first heavy chain having the sequence of SEQ ID NO: 5, SEQ ID NO: 33 or SEQ ID NO: 36, optionally comprising one or more amino acid substitutions in the heavy chain constant region; (b) a second heavy chain derived from a different antibody clone to the first heavy chain; and (c) a first and second light chain having the sequence of SEQ ID NO: 15, optionally comprising one or more amino acid substitutions in the light chain constant region.

In some embodiments, the bispecific antibody comprises: (a) a first heavy chain having the sequence of SEQ ID NO: 5, SEQ ID NO: 33 or SEQ ID NO: 36, optionally comprising one or more amino acid substitutions in the heavy chain constant region; (b) a second heavy chain derived from a different antibody clone to the first heavy chain; and a first and second light chain having the sequence of SEQ ID NO: 20, optionally comprising one or more amino acid substitutions in the light chain constant region.

In some embodiments, the bispecific antibody comprises: (a) a first heavy chain having the sequence of SEQ ID NO: 5, SEQ ID NO: 33 or SEQ ID NO: 36, optionally comprising one or more amino acid substitutions in the heavy chain constant region; (b) a second heavy chain derived from a different antibody clone to the first heavy chain; and (c) a first and second light chain having the sequence of SEQ ID NO: 25, optionally comprising one or more amino acid substitutions in the light chain constant region.

The antibody of the invention may further comprise constant regions. Preferably, the constant regions are of human origin. In some embodiments, the heavy chain constant region may be an IgA, IgD, IgE, IgG or IgM constant region. Preferably, the constant region is an IgG constant region, for example, it may be IgG1, IgG2, IgG3 or IgG4. In a preferred embodiment, the constant region is an IgG1 constant region. The light chain constant region may be either a lambda or a kappa constant region. Preferably, the light chain constant region is a kappa constant region.

In a preferred embodiment, the heavy chain constant region comprises one of more or all of a CH1, CH2 and/or CH3 domain and the light chain constant region comprises a CL domain.

In some embodiments, the antibody comprises: (a) a heavy chain of SEQ ID NO: 5, SEQ ID NO: 33 or SEQ ID NO: 36 and (b) a light chain of SEQ ID NO: 10, 15, 20 or 25, wherein the heavy and/or light chain constant domains further comprise one or more amino acid substitutions. For example, the constant domains may comprise amino acid substitutions which enhance binding of the Fc region to one or more Fc receptors, increase or reduce one or more Fc effector functions, increase or decrease the half-life of the antibody, improve the manufacturability of the antibody, promote heterodimerisation of the antibody heavy chains/and or light chains, or any combination thereof.

Alternatively or in addition, the variable domains may comprise amino acid substitutions which improve the binding affinity of the antibody for one or more variants of SARS-COV-2.

In preferred embodiments, the antibody further comprises an Fc region. Preferably, the Fc region is an IgG1 Fc region. In some embodiments, the Fc region may be a modified IgG1 Fc region comprising one or more amino acid substitutions relative to the wildtype IgG1 Fc region. In preferred embodiments, the amino acid substitutions increase the half-life of the antibody relative to the wildtype IgG1 Fc region.

In certain embodiments, the amino acid substitutions used to improve the half-life of an IgG1 antibody are selected from M252Y/S254T/T256E (‘YTE’), M428L/N434S (‘LS’), S239D/1332E (‘DE’), M252Y/T256D (‘YD’), T256D/T307Q (‘DQ’) and/or T256D/T307W (‘DW’) or any combination thereof (numbered according to EU numbering). In preferred embodiments, the amino acid substitutions M252Y/S254T/T256E (‘YTE’) may be used to improve the half-life of an IgG1 antibody.

In other embodiments, the one or more substitution(s) may remove or enhance binding of the Fc region to an Fc receptor, increase or eliminate an effector function such as antibody-dependent cellular cytotoxicity (ADCC) and/or complement-dependent cytotoxicity (CDC), potentiate immune complex formation, or any combination thereof. The one or more substitution(s) may be any suitable substitutions known in the art.

In other embodiments, the one or more substitution(s) may promote heterodimerisation of the antibody heavy chains/and or light chains. For example, where the antibody of the invention is a bispecific antibody, the Fc region of the first heavy chain and the Fc region of the second heavy chain may each comprise one or more substitution(s) which promote selective association of the first heavy chain and the second heavy chain.

Alternatively, or in addition, the CH1 region of the first heavy chain and the CL region of the first light chain may each comprise one or more substitution(s) which promote selective association of the first heavy chain with the first light chain and/or the CH1 region of the second heavy chain and the CL region of the second light chain may each comprise one or more substitution(s) which promote selective association of the first heavy chain with the first light chain.

In a further aspect, the invention provides a polynucleotide encoding the antibody according to the invention, a vector comprising said polynucleotide, or a host cell comprising said vector.

In a further aspect, the invention provides a pharmaceutical composition comprising an antibody according to the invention and at least one pharmaceutically acceptable diluent or carrier. Optionally, the pharmaceutical composition may also contain one or more further antibodies capable of binding to SARS-COV-2. Alternatively or in addition, the pharmaceutical composition may also contain one or more additional therapeutic agents, such as an anti-inflammatory agent or an antiviral agent.

In some embodiments, the invention provides a pharmaceutical composition comprising one or more antibodies according to the present invention. For example, the composition may comprise antibodies one, two, thee or all four of the antibodies in Table 2.

The antibody of the invention may be able to provide in vivo protection against coronavirus (e.g. SARS-COV-2) in infected subjects. For example, administration of an antibody of the invention to a subject infected with a coronavirus (e.g. SARS-COV-2) may result in prevention of infection, an improved chance of survival, a reduction in the duration of illness, and/or a reduction in severity of symptoms.

In some embodiments, an antibody of the invention may be used for the prevention of infection prior to exposure to SARS-COV-2 (pre-exposure prophylaxis). In some embodiments, an antibody of the invention may be used for prevention of infection after a recent exposure to SARS-COV-2 (post exposure prophylaxis). In some embodiments an antibody of the invention may be used for treatment of an acute SARS-COV-2 infection (COVID-19). In some embodiments, an antibody of the invention may be used for treatment of the long-term effects of SARS-COV-2 infection (Long COVID).

Accordingly, in a further aspect, the invention provides an antibody or pharmaceutical composition for use in a method of treating, preventing or diagnosing a disease or a complication associated with coronavirus (e.g. SARS-COV-2) infection.

In one embodiment, the antibody or the pharmaceutical composition of the invention is for use in a method of treating, preventing or diagnosing coronavirus infections caused by a SARS-CoV2 variant. Examples of SARS-COV-2 variants which can be treated with one or more antibodies of the present invention include but are not limited to hCoV-19/Wuhan/WIV04/2019, SARS-COV-2/human/AUS/VIC01/2020, A.23.1, B.1.1.7 (Alpha), B.1.351 (Beta), B.1.258, B.1.526.2, B.1.616, B.1.617.1, B.1.617.2 (Delta), C36.3, C.37 or P.1 (Gamma).

In a preferred embodiment, the antibody or the pharmaceutical composition of the invention is for use in a method of treating, preventing or diagnosing COVID-19 infections caused by the hCoV-19/Wuhan/WIV04/2019 variant, SARS-COV-2/human/AUS/VIC01/2020 variant, Alpha (B.1.1.7) variant, Beta (B.1.351) variant, Gamma (P1) variant, Delta (B1.617.2) variant, another variant comprising mutations in the RBD of the spike protein of SARS-COV-2 at one or more of positions 501, 484, 452, 478 and/or 417, and/or any other variant of concern.

The skilled person would appreciate that the above list is not exhaustive and that the antibodies or pharmaceutical compositions of the invention may be suitable for use in a method of treating, preventing or diagnosing coronavirus infections caused by any other known or yet-to-be identified SARS-COV-2 variant, including any variant for which the virus genotype is listed in SARS-COV-2 databases, any variant which is known to exist in nature, any variant which has been generated in laboratory experiments or any variant which has been generated through infection of susceptible animals. Examples of variants which can be treated, prevented or diagnosed with antibodies according to the invention are listed in the SARS-COV-2 genome and spike databases.

For example, the antibodies according to the invention may be for use in a method of treating, preventing or diagnosing coronavirus infections caused by a SARS-COV2 variant having genetic variation within the spike gene relative to the hCoV-19/Wuhan/WIV04/2019 variant. In some embodiments, the spike protein of the variant may be more than 80%, more than 85%, more 90%, more than 91%, more than 92%, more than 93%, more than 94%, more than 95%, more than 96%, more than 97%, more than 98%, or more than 99% identical to that of hCoV-19/Wuhan/WIV04/2019. For example, the spike protein of the variant may differ from the spike protein of hCoV-19/Wuhan/WIV04/2019 by one, two, three, four, five, more than five, or up to 10 amino acids. In some embodiments, the mutations are mutations that have been observed in a naturally occurring variant of SARS-COV-2. In other embodiments, the mutations are mutations that have not been seen in a naturally occurring variant of SARS-COV-2 but have been engineered in the laboratory. In preferred embodiments, the mutations are mutations in the RBD of the spike protein of SARS-COV-2 at one or more of positions 501, 484, 452, 478 and/or 417.

In a further aspect, the invention provides a method of treating a subject comprising administering a therapeutically effective amount of an antibody according to the invention or a pharmaceutical composition according to the invention to said subject. Further provided is an antibody according to the invention for use in the manufacture of a medicament for treating a subject.

In a further aspect, the invention provides a method for producing an antibody that is capable of binding to the spike protein of SARS-COV-2, the method comprising culturing the host cell of the invention under optimal conditions for the production of an antibody according to the invention and isolating the antibody from said culture.

In a further aspect, the invention provides a method of identifying the presence of a coronavirus (e.g. SARS-COV-2) or a component or fragment thereof in a sample, the method comprising contacting the sample with an antibody according to the invention and determining whether the antibody forms an antibody-antigen complex, wherein the formation of the antibody-antigen complex indicates that the sample is positive for coronavirus (e.g. SARS-COV-2).

In some embodiments, the antibodies of the invention do not comprise an N-glycosylation site in the heavy chain variable region and/or the light chain variable region. In one embodiment, the antibodies of the invention do not comprise an N-glycosylation site in the heavy chain variable region. Preferably, the antibodies of the invention do not comprise an N-glycosylation site in the VHCDR3.

The binding affinity of the antibodies according to the invention for the spike protein of SARS-CoV-2 may be quantified by determining the equilibrium dissociation constant (K_{D}). In some embodiments, the antibodies of the invention bind to the spike protein of a SARS-COV-2 variant with a K_{D} value of less than 5 nM, less than 4 nM, less than 3 nM, less than 2 nM, less than 1 nM, less than 0.5 nM, less than 0.4 nM, less than 0.3 nM, less than 0.2 nM or less than 0.1 nM. The K_{D} value can be measured by any suitable means known in the art, for example, by ELISA or Surface Plasmon Resonance (Biacore). In a preferred embodiment, the SARS-COV-2 variant tested is SARS-COV-2/human/AUS/VIC01/2020 or 19/Wuhan/WIV04/2019.

In further embodiments, the variant tested is the Alpha (B.1.1.7) variant, Beta (B.1.351) variant, Gamma (P1) variant, Delta (B1.617.2) variant, another variant comprising mutations in the RBD of the spike protein of SARS-COV-2 at one or more of positions 501, 484, 452, 478 and/or 417, and/or another variant of concern.

In one embodiment, the antibodies of the invention totally or partially block interactions between the spike protein of SARS-COV-2 and ACE2, the cell surface receptor which mediates target cell fusion. For example, the antibodies of the invention may block interaction directly or may indirectly interfere with fusion by disrupting the conformation of the spike protein. In some embodiments, the antibodies of the invention may reduce spike-ACE2 interaction by more than 50%, more than 60%, more than 70%, more than 80%, more than 90%, more than 95%, more than 99% or by 100%. Blocking of spike-ACE2 formation can be measured by any suitable means known in the art, for example, by ELISA.

In one embodiment, the antibodies of the invention may be able to neutralise at least one biological activity of SAR-COV-2. For example, an antibody of the invention may be able to neutralise viral infectivity. Suitable methods of measuring viral infectivity are known in the art. For example, the ability of an antibody to neutralise viral infectivity may be measured with a focus reduction neutralisation assay (FRNT) or any other appropriate method.

Neutralisation of SARS-COV-2 can be measured using half-maximal inhibitory concentration (IC₅₀) values. In some embodiments, the antibody according to the invention may have an IC₅₀ value of less than 0.1 μg/ml, less than 0.05 μg/ml, less than 0.01 μg/ml, less than 0.005 μg/ml, less than 0.002 μg/ml or less than 0.001 μg/ml. In some instances, an antibody of the invention may have an IC₅₀ value of between 0.0001 μg/ml and 0.1 μg/ml, between 0.0001 μg/ml and 0.05 μg/ml or between 0.0001 μg/ml and 0.001 μg/ml against one or more SAR-COV-2 variants.

In a preferred embodiment, the antibody according to the invention may have an IC₅₀ value of less than 0.1 μg/ml, less than 0.05 μg/ml, less than 0.01 μg/ml, less than 0.005 μg/ml, less than 0.002 μg/ml or less than 0.001 μg/ml against the hCoV-19/Wuhan/WIV04/2019 variant, SARS-CoV-2/human/AUS/VIC01/2020 variant, Alpha (B.1.1.7) variant, Beta (B.1.351) variant, Gamma (P1) variant, Delta (B1.617.2) variant, another variant comprising mutations in the RBD of the spike protein of SARS-COV-2 at one or more of positions 501, 484, 452, 478 and/or 417, and/or another variant of concern.

In a particularly preferred embodiment, the IC₅₀ value is less than 0.01 μg/ml or less than 0.02 μg/ml against the hCoV-19/Wuhan/WIV04/2019 variant, SARS-COV-2/human/AUS/VIC01/2020 variant, Alpha (B.1.1.7) variant, Beta (B.1.351) variant, Gamma (P1) variant, Delta (B1.617.2) variant, another variant comprising mutations in the RBD of the spike protein of SARS-COV-2 at one or more of positions 501, 484, 452, 478 and/or 417, and/or another variant of concern.

In some instances an antibody of the invention may have an IC₅₀ value of between 0.0001 μg/ml and 0.1 μg/ml, between 0.0001 μg/ml and 0.05 μg/ml or between 0.0001 μg/ml and 0.001 μg/ml against the hCoV-19/Wuhan/WIV04/2019 variant, SARS-COV-2/human/AUS/VIC01/2020 variant, Alpha (B.1.1.7) variant, Beta (B.1.351) variant, Gamma (P1) variant, Delta (B1.617.2) variant, another variant comprising mutations in the RBD of the spike protein of SARS-COV-2 at one or more of positions 501, 484, 452, 478 and/or 417, and/or any other variant of concern.

Neutralisation of SARS-COV-2 can also be measured using 80% maximal inhibitory concentration (IC₈₀) values. In some embodiments, the antibody according to the invention may have an IC₈₀ value of less than 0.1 μg/ml, less than 0.05 μg/ml, less than 0.01 μg/ml, less than 0.005 μg/ml, less than 0.002 μg/ml or less than 0.001 μg/ml. In some instances, an antibody of the invention may have an IC₅₀ value of between 0.0001 μg/ml and 0.1 μg/ml, between 0.0001 μg/ml and 0.05 μg/ml or between 0.0001 μg/ml and 0.001 μg/ml against one or more SAR-COV-2 variants.

In a preferred embodiment, the antibody according to the invention may have an IC₈₀ value of less than 0.1 μg/ml, less than 0.05 μg/ml, less than 0.01 μg/ml, less than 0.005 μg/ml, less than 0.002 μg/ml or less than 0.001 μg/ml against the hCoV-19/Wuhan/WIV04/2019 variant, SARS-CoV-2/human/AUS/VIC01/2020 variant, Alpha (B.1.1.7) variant, Beta (B.1.351) variant, Gamma (P1) variant, Delta (B1.617.2) variant, another variant comprising mutations in the RBD of the spike protein of SARS-COV-2 at one or more of positions 501, 484, 452, 478 and/or 417, and/or another variant of concern.

In a particularly preferred embodiment, the IC₈₀ value is less than 0.03 μg/ml or less than 0.06 μg/ml against the hCoV-19/Wuhan/WIV04/2019 variant, SARS-COV-2/human/AUS/VIC01/2020 variant, Alpha (B.1.1.7) variant, Beta (B.1.351) variant, Gamma (P1) variant, Delta (B1.617.2) variant, another variant comprising mutations in the RBD of the spike protein of SARS-COV-2 at one or more of positions 501, 484, 452, 478 and/or 417, and/or another variant of concern.

In some instances an antibody of the invention may have an IC₈₀ value of between 0.0001 μg/ml and 0.1 μg/ml, between 0.0001 μg/ml and 0.05 μg/ml or between 0.0001 μg/ml and 0.001 μg/ml against the hCoV-19/Wuhan/WIV04/2019 variant, SARS-COV-2/human/AUS/VIC01/2020 variant, Alpha (B.1.1.7) variant, Beta (B.1.351) variant, Gamma (P1) variant, Delta (B1.617.2) variant, another variant comprising mutations in the RBD of the spike protein of SARS-COV-2 at one or more of positions 501, 484, 452, 478 and/or 417, and/or any other variant of concern.

### BRIEF DESCRIPTION OF THE FIGURES

FIG. 1 shows neutralisation plots for RQCov-05/02, RQCov-05/03, RQCov-05/04 and RQCov-05/05 monoclonal antibodies against SARS-COV-2 Victoria, Alpha, Beta, Gamma and Delta variants. Data are shows as Log antibody concentration (ug/ml) plotted on X-axis versus % neutralisation plotted on Y-axis.

FIG. 2 shows a neutralisation plot for RQCov-06/03 monoclonal antibodies against SARS-CoV-2 Victoria, Alpha, Beta, Gamma and Delta variants. Data are shows as Log antibody concentration (ug/ml) plotted on X-axis versus % neutralisation plotted on Y-axis.

FIG. 3 shows neutralisation plots for RQCov-07/02, RQCov-07/03 and RQCov-07/07 monoclonal antibodies against SARS-COV-2 Victoria, Alpha, Beta, Gamma and Delta variants. Data are shows as Log antibody concentration (ug/ml) plotted on X-axis versus % neutralisation plotted on Y-axis.

### DETAILED DESCRIPTION

The antibodies of the invention specifically bind to the spike protein of SARS-COV-2. Preferably, the antibodies of the invention bind to the receptor binding domain (RBD) of the S1 subunit of the spike protein of SARS-COV-2. Alternatively, the antibodies of the invention may bind to a quaternary epitope that arises though binding higher order structures constituting the trimeric spike protein.

An antibody of the invention may comprise at least three, four, five, or six CDRs from Table 1. For example, the antibody may comprise at least one, at least two or all three heavy chain CDRs (VHCDRs) and/or at least one, at least two or all three light chain CDRs (VLCDRs) from an antibody in Table 1. Preferably, the antibody comprises six CDRs from Table 1 (i.e. all three heavy CDRs and all three light chain CDRs).

In one embodiment, an antibody of the invention may comprise a VHCDR1, VHCDR2 and VHCDR3 having the amino acid sequences of SEQ ID NOs: 1, 2 and 3, respectively and a VLCDR1, VLCDR2 and VLCDR3 having (a) the amino acid sequences of SEQ ID NOs: 6, 7 and 8, respectively; (b) the amino acid sequences of SEQ ID NOs: 11, 12 and 13, respectively; (c) the amino acid sequences of SEQ ID NOs: 16, 17 and 18, respectively; or (d) the amino acid sequences of SEQ ID NOs: 21, 22 and 23, respectively.

In one embodiment, an antibody of the invention may comprise a VHCDR1, VHCDR2 and VHCDR3 having the amino acid sequences of SEQ ID NOs: 1, 2 and 31, respectively and a VLCDR1, VLCDR2 and VLCDR3 having (a) the amino acid sequences of SEQ ID NOs: 6, 7 and 8, respectively; (b) the amino acid sequences of SEQ ID NOs: 11, 12 and 13, respectively; (c) the amino acid sequences of SEQ ID NOs: 16, 17 and 18, respectively; or (d) the amino acid sequences of SEQ ID NOs: 21, 22 and 23, respectively.

In one embodiment, an antibody of the invention may comprise a VHCDR1, VHCDR2 and VHCDR3 having the amino acid sequences of SEQ ID NOs: 1, 2 and 34, respectively and a VLCDR1, VLCDR2 and VLCDR3 having (a) the amino acid sequences of SEQ ID NOs: 6, 7 and 8, respectively; (b) the amino acid sequences of SEQ ID NOs: 11, 12 and 13, respectively; (c) the amino acid sequences of SEQ ID NOs: 16, 17 and 18, respectively; or (d) the amino acid sequences of SEQ ID NOs: 21, 22 and 23, respectively.

In one embodiment, an antibody of the invention may comprise: (a) a heavy chain variable region comprising a VHCDR1 comprising the amino acid sequence of SEQ ID NO: 1, a VHCDR2 comprising the amino acid sequence of SEQ ID NO: 2; and a VHCDR3 comprising the amino acid sequence of SEQ ID NO: 3, SEQ ID NO: 31 or SEQ ID NO: 34; and/or (b) a light chain variable region comprising a VLCDR1 comprising the amino acid sequence of SEQ ID NO: 6, a VLCDR2 comprising the amino acid sequence of SEQ ID NO: 7; and a VLCDR3 comprising the amino acid sequence of SEQ ID NO: 8.

In one embodiment, an antibody of the invention may comprise: (a) a heavy chain variable region comprising a VHCDR1 comprising the amino acid sequence of SEQ ID NO: 1, a VHCDR2 comprising the amino acid sequence of SEQ ID NO 2; and a VHCDR3 comprising the amino acid sequence of SEQ ID NO 3, SEQ ID NO: 31 or SEQ ID NO: 34; and/or (b) a light chain variable region comprising a VLCDR1 comprising the amino acid sequence of SEQ ID NO: 11, a VLCDR2 comprising the amino acid sequence of SEQ ID NO: 12; and a VLCDR3 comprising the amino acid sequence of SEQ ID NO: 13.

In one embodiment, an antibody of the invention may comprise: (a) a heavy chain variable region comprising a VHCDR1 comprising the amino acid sequence of SEQ ID NO: 1, a VHCDR2 comprising the amino acid sequence of SEQ ID NO: 2; and a VHCDR3 comprising the amino acid sequence of SEQ ID NO: 3, SEQ ID NO: 31 or SEQ ID NO: 34; and/or (b) a light chain variable region comprising a VLCDR1 comprising the amino acid sequence of SEQ ID NO: 16, a VLCDR2 comprising the amino acid sequence of SEQ ID NO: 17; and a VLCDR3 comprising the amino acid sequence of SEQ ID NO: 18.

In one embodiment, an antibody of the invention may comprise: (a) a heavy chain variable region comprising a VHCDR1 comprising the amino acid sequence of SEQ ID NO: 1, a VHCDR2 comprising the amino acid sequence of SEQ ID NO: 2; and a VHCDR3 comprising the amino acid sequence of SEQ ID NO: 3, SEQ ID NO: 31 or SEQ ID NO: 34; and/or (b) a light chain variable region comprising a VLCDR1 comprising the amino acid sequence of SEQ ID NO: 21, a VLCDR2 comprising the amino acid sequence of SEQ ID NO: 22; and a VLCDR3 comprising the amino acid sequence of SEQ ID NO: 23.

In a further embodiment, the antibody of the invention may comprise a heavy chain variable domain comprising or consisting of an amino acid sequence having at least 80% sequence identity to the heavy chain variable domain of an antibody in Table 1. Alternatively or in addition, an antibody of the invention may comprise a light chain variable domain comprising or consisting of an amino acid sequence having at least 80% sequence identity to a light chain variable domain of an antibody in Table 1.

In one embodiment, an antibody of the invention may comprise a heavy chain variable domain comprising or consisting of an amino acid sequence having more than 80%, more than 85%, more than 90%, more than 95%, more than 96%, more than 97%, more than 98%, more than 99% or 100% sequence identity to SEQ ID NO: 4, SEQ ID NO: 32 or SEQ ID NO: 35. In one embodiment, an antibody of the invention may comprise a light chain variable domain comprising or consisting of an amino acid sequence having more than 80%, more than 85%, more than 90%, more than 95%, more than 96%, more than 97%, more than 98%, more than 99% or 100% sequence identity to SEQ ID NO: 9, 14, 19 or 24.

In one embodiment, an antibody of the invention may comprise a heavy chain comprising or consisting of an amino acid sequence having more than 80%, more than 85%, more than 90%, more than 95%, more than 96%, more than 97%, more than 98%, more than 99% or 100% sequence identity to SEQ ID NO: 5, SEQ ID NO: 33 or SEQ ID NO: 36. In one embodiment, an antibody of the invention may comprise a light chain comprising or consisting of an amino acid sequence having more than 80%, more than 85%, more than 90%, more than 95%, more than 96%, more than 97%, more than 98%, more than 99% or 100% sequence identity to SEQ ID NO: 10, 15, 20 or 25.

The antibody of the invention may be derived from an antibody or antibodies identified from a recovered SARS-COV-2 COVID-19 patient (see e.g Dejnirattisai et al. “The antigenic anatomy of SARS-COV-2 receptor binding domain.” Cell 184.8 (2021): 2183-2200 and Dejnirattisai et al. “Antibody evasion by the P. 1 variant of SARS-COV-2.” Cell 184.11 (2021): 2939-2954).

Preferably, the antibody or antibodies from which the antibodies of the present invention are derived retain strong neutralisation ability against multiple SARS-COV-2 variants i.e. they are effective against two or more SARS-COV-2 variants.

For example, they may have an IC₅₀ value of less than 0.1 μg/ml, less than 0.05 μg/ml, less than 0.01 μg/ml, less than 0.005 μg/ml, less than 0.002 μg/ml, or less than 0.001 μg/ml. In some instances an antibody of the invention may have an IC₅₀ value of between 0.0001 μg/ml and 0.1 μg/ml, between 0.0001 μg/ml and 0.05 μg/ml or between 0.0001 μg/ml and 0.001 μg/ml against at least two, at least three, at least four, or at least five or more different SARS-COV-2 variants.

The antibodies of the invention may be mixed-chain antibodies. In one embodiment, an antibody of the invention may comprise a heavy chain variable domain derived from the antibody RQCov-01. In one embodiment, an antibody of the invention may comprise a light chain variable domain derived from the antibody RQCov-02, RQCov-03 or RQCov-04. In one embodiment, the antibody of the invention may comprise a heavy chain derived from RQCov-01, and a light chain derived from RQCov-02. In one embodiment, the antibody of the invention may comprise a heavy chain derived from RQCov-01, and a light chain derived from RQCov-03. In one embodiment, the antibody of the invention may comprise a heavy chain derived from RQCov-01, and a light chain derived from RQCov-04. In one embodiment, the antibody of the invention may comprise a heavy chain and a light chain derived from RQCov-01.

In some embodiments, the heavy chain variable domain derived from the antibody RQCov-01 is the heavy chain variable domain of RQCov-05. In some embodiments, the heavy chain variable domain derived from the antibody RQCov-01 is the heavy chain variable domain of RQCov-06. In some embodiments, the heavy chain variable domain derived from the antibody RQCov-01 is the heavy chain variable domain of RQCov-07. The heavy chain of RQCov-05 RQCov-06 and RQCov-07 have been modified such that it does not contain a N-glycosylation site in the heavy chain variable region in VHCDR3.

The heavy chain domains of each of RQCov-01, RQCov-02, RQCov-03, RQCov-04, RQCov-05, RQCov-06 and RQCov-07 are derived from an IGHV1-58 v-region. It has been found that switching the heavy chains and light chains between different monoclonal antibodies which have heavy chain variable domains derived from the same v-region results in antibodies that are particularly useful for neutralising SARS-COV-2.

In preferred embodiments, the antibodies of the present invention have one of the heavy and light chain combinations provided in Table 2.

TABLE 1
Sequences of the antibodies of the invention

| SEQ ID NO: | Sequence | Description |
|---|---|---|
| 1 | GFTFITSA | RQCov-05 VHCDR1 |
| | | RQCov-06 VHCDR1 |
| | | RQCov-07 VHCDR1 |
| 2 | IAVGSGNT | RQCov-05 VHCDR2 |
| | | RQCov-06 VHCDR2 |
| | | RQCov-07 VHCDR2 |
| 3 | AAPHCDRTSCHDGFDI | RQCov-05 VHCDR3 |
| 4 | QVQLVESGPEMKKPGTSVKVSCKASGFTFIT | RQCov-05 VH |
| | SAVQWVRQARGQRLEWMGWIAVGSGNTNY | |
| | AQKFQDRVTINRDMSTSTAYMELSSLRSEDT | |
| | AVYYCAAPHCDRTSCHDGFDIWGQGTMVTV | |
| | SS | |
| 5 | QVQLVESGPEMKKPGTSVKVSCKASGFTFIT | RQCov-05 Heavy chain |
| | SAVQWVRQARGQRLEWMGWIAVGSGNTNY | |
| | AQKFQDRVTINRDMSTSTAYMELSSLRSEDT | |
| | AVYYCAAPHCDRTSCHDGFDIWGQGTMVTV | |
| | SSASTKGPSVFPLAPSSKSTSGGTAALGCLV | |
| | KDYFPEPVTVSWNSGALTSGVHTFPAVLQS | |
| | SGLYSLSSVVTVPSSSLGTQTYICNVNHKPS | |
| | NTKVDKKVEPKSCDKTHTCPPCPAPELLGGP | |
| | SVFLFPPKPKDTLMISRTPEVTCVVVDVSHE | |
| | DPEVKFNWYVDGVEVHNAKTKPREEQYNST | |
| | YRVVSVLTVLHQDWLNGKEYKCKVSNKALP | |
| | APIEKTISKAKGQPREPQVYTLPPSREEMTK | |
| | NQVSLTCLVKGFYPSDIAVEWESNGQPENN | |
| | YKTTPPVLDSDGSFFLYSKLTVDKSRWQQG | |
| | NVFSCSVMHEALHNHYTQKSLSLSPGK | |
| 6 | QSVSSSY | RQCov-02 VLCDR1 |
| 7 | GAS | RQCov-02 VLCDR2 |
| 8 | QQYGSSPWT | RQCov-02 VLCDR3 |
| 9 | DIQMTQSPGTLSLSPGERATLSCRASQSVSS | RQCov-02 VL |
| | SYLAWYQQKPGQAPRLLIYGASSRATGIPDR | |
| | FSGSGSGTDFTLTISRLEPEDFGVYYCQQYG | |
| | SSPWTFGQGTKVEIK | |
| 10 | DIQMTQSPGTLSLSPGERATLSCRASQSVSS | RQCov-02 Light chain |
| | SYLAWYQQKPGQAPRLLIYGASSRATGIPDR | |
| | FSGSGSGTDFTLTISRLEPEDFGVYYCQQYG | |
| | SSPWTFGQGTKVEIKRTVAAPSVFIFPPSDE | |
| | QLKSGTASVVCLLNNFYPREAKVQWKVDNA | |
| | LQSGNSQESVTEQDSKDSTYSLSSTLTLSKA | |
| | DYEKHKVYACEVTHQGLSSPVTKSFNRGEC | |
| 11 | QSVRSSY | RQCov-03 VLCDR1 |
| 12 | GAS | RQCov-03 VLCDR2 |
| 13 | QQYGSSPWT | RQCov-03 VLCDR3 |
| 14 | DIVMTQSPGTLSLSPGERATLSCRASQSVRS | RQCov-03 VL |
| | SYLAWYQQKPGQAPRLLIYGASRRGTGIPDR | |
| | FSGSGSGTDFTLTISRLEPEDFAVYYCQQYG | |
| | SSPWTFGQGTKVEIK | |
| 15 | DIVMTQSPGTLSLSPGERATLSCRASQSVRS | RQCov-03 Light chain |
| | SYLAWYQQKPGQAPRLLIYGASRRGTGIPDR | |
| | FSGSGSGTDFTLTISRLEPEDFAVYYCQQYG | |
| | SSPWTFGQGTKVEIKRTVAAPSVFIFPPSDE | |
| | QLKSGTASVVCLLNNFYPREAKVQWKVDNA | |
| | LQSGNSQESVTEQDSKDSTYSLSSTLTLSKA | |
| | DYEKHKVYACEVTHQGLSSPVTKSFNRGEC | |
| 16 | QSVSSSY | RQCov-04 VLCDR1 VL |
| 17 | GAS | RQCov-04 VLCDR2 VL |
| 18 | QQYGSSPFT | RQCov-04 VLCDR3 VL |
| 19 | EIVMTQSPGTLSLSPGERATLSCRASQSVSS | RQCov-04 VL |
| | SYLAWYQQKPGQAPRLLIYGASSRATGIPDR | |
| | FSGSGSGTDFTLTISRLEPEDFAVYYCQQYG | |
| | SSPFTFGGGTKVEIK | |
| 20 | EIVMTQSPGTLSLSPGERATLSCRASQSVSS | RQCov-04 Light chain |
| | SYLAWYQQKPGQAPRLLIYGASSRATGIPDR | |
| | FSGSGSGTDFTLTISRLEPEDFAVYYCQQYG | |
| | SSPFTFGGGTKVEIKRTVAAPSVFIFPPSDEQ | |
| | LKSGTASVVCLLNNFYPREAKVQWKVDNAL | |
| | QSGNSQESVTEQDSKDSTYSLSSTLTLSKAD | |
| | YEKHKVYACEVTHQGLSSPVTKSFNRGEC | |
| 21 | QSVSRNY | RQCov-01 VLCDR1 |
| | | RQCov-05 VLCDR1 |
| | | RQCov-06 VLCDR1 |
| | | RQCov-07 VLCDR1 |
| 22 | GAS | RQCov-01 VLCDR2 VL |
| | | RQCov-05 VLCDR2 VL |
| | | RQCov-06 VLCDR2 VL |
| | | RQCov-07 VLCDR2 VL |
| 23 | QQYGSSLFT | RQCov-01 VLCDR3 VL |
| | | RQCov-05 VLCDR3 VL |
| | | RQCov-06 VLCDR3 VL |
| | | RQCov-07 VLCDR3 VL |
| 24 | EIVLTQSPGTLSLSPGERATLSCRASQSVSR | RQCov-01 VL |
| | NYLAWYQQKPGQVPRLLIYGASSRATGIPDR | RQCov-05 VL |
| | FRGSGSGTDFTLTINRLESEDFAVYYCQQYG | RQCov-06 VL |
| | SSLFTFGPGTKVDIK | RQCov-07 VL |
| 25 | EIVLTQSPGTLSLSPGERATLSCRASQSVSR | RQCov-01 Light chain |
| | NYLAWYQQKPGQVPRLLIYGASSRATGIPDR | RQCov-05 Light chain |
| | FRGSGSGTDFTLTINRLESEDFAVYYCQQYG | RQCov-06 Light chain |
| | SSLFTFGPGTKVDIKRTVAAPSVFIFPPSDEQ | RQCov-07 Light chain |
| | LKSGTASVVCLLNNFYPREAKVQWKVDNAL | |
| | QSGNSQESVTEQDSKDSTYSLSSTLTLSKAD | |
| | YEKHKVYACEVTHQGLSSPVTKSFNRGEC | |
| 26 | EPKSCDKTHTCPPCPAPELLGGPSVFLFPPK | IgG1 Fc |
| | PKDTLMISRTPEVTCVVVDVSHEDPEVKFNW | |
| | YVDGVEVHNAKTKPREEQYNSTYRVVSVLT | |
| | VLHQDWLNGKEYKCKVSNKALPAPIEKTISK | |
| | AKGQPREPQVYTLPPSREEMTKNQVSLTCL | |
| | VKGFYPSDIAVEWESNGQPENNYKTTPPVLD | |
| | SDGSFFLYSKLTVDKSRWQQGNVFSCSVMH | |
| | EALHNHYTQKSLSLSPGK | |
| 27 | GFTFITSA | RQCov-01 VHCDR1 |
| 28 | IAVGSGNT | RQCov-01 VHCDR2 |
| 29 | AAPHCNRTSCHDGFDI | RQCov-01 VHCDR3 |
| 30 | QVQLVESGPEMKKPGTSVKVSCKASGFTFIT | RQCov-01 VH |
| | SAVQWVRQARGQRLEWMGWIAVGSGNTNY | |
| | AQKFQDRVTINRDMSTSTAYMELSSLRSEDT | |
| | AVYYCAAPHCNRTSCHDGFDIWGQGTMVTV | |
| | SS | |
| 31 | AAPHCSRTSCHDGFDI | RQCov-07 VHCDR3 |
| 32 | QVQLVESGPEMKKPGTSVKVSCKASGFTFIT | RQCov-07 VH |
| | SAVQWVRQARGQRLEWMGWIAVGSGNTNY | |
| | AQKFQDRVTINRDMSTSTAYMELSSLRSEDT | |
| | AVYYCAAPHCSRTSCHDGFDIWGQGTMVTV | |
| | SS | |
| 33 | QVQLVESGPEMKKPGTSVKVSCKASGFTFIT | RQCov-07 Heavy chain |
| | SAVQWVRQARGQRLEWMGWIAVGSGNTNY | |
| | AQKFQDRVTINRDMSTSTAYMELSSLRSEDT | |
| | AVYYCAAPHCSRTSCHDGFDIWGQGTMVTV | |
| | SSASTKGPSVFPLAPSSKSTSGGTAALGCLV | |
| | KDYFPEPVTVSWNSGALTSGVHTFPAVLQS | |
| | SGLYSLSSVVTVPSSSLGTQTYICNVNHKPS | |
| | NTKVDKKVEPKSCDKTHTCPPCPAPELLGGP | |
| | SVFLFPPKPKDTLMISRTPEVTCVVVDVSHE | |
| | DPEVKFNWYVDGVEVHNAKTKPREEQYNST | |
| | YRVVSVLTVLHQDWLNGKEYKCKVSNKALP | |
| | APIEKTISKAKGQPREPQVYTLPPSREEMTK | |
| | NQVSLTCLVKGFYPSDIAVEWESNGQPENN | |
| | YKTTPPVLDSDGSFFLYSKLTVDKSRWQQG | |
| | NVFSCSVMHEALHNHYTQKSLSLSPGK | |
| 34 | AAPHCQRTSCHDGFDI | RQCov-06 VHCDR3 |
| 35 | QVQLVESGPEMKKPGTSVKVSCKASGFTFIT | RQCov-06 VH |
| | SAVQWVRQARGQRLEWMGWIAVGSGNTNY | |
| | AQKFQDRVTINRDMSTSTAYMELSSLRSEDT | |
| | AVYYCAAPHCQRTSCHDGFDIWGQGTMVTV | |
| | SS | |
| 36 | QVQLVESGPEMKKPGTSVKVSCKASGFTFIT | RQCov-06 Heavy chain |
| | SAVQWVRQARGQRLEWMGWIAVGSGNTNY | |
| | AQKFQDRVTINRDMSTSTAYMELSSLRSEDT | |
| | AVYYCAAPHCQRTSCHDGFDIWGQGTMVTV | |
| | SSASTKGPSVFPLAPSSKSTSGGTAALGCLV | |
| | KDYFPEPVTVSWNSGALTSGVHTFPAVLQS | |
| | SGLYSLSSVVTVPSSSLGTQTYICNVNHKPS | |
| | NTKVDKKVEPKSCDKTHTCPPCPAPELLGGP | |
| | SVFLFPPKPKDTLMISRTPEVTCVVVDVSHE | |
| | DPEVKFNWYVDGVEVHNAKTKPREEQYNST | |
| | YRVVSVLTVLHQDWLNGKEYKCKVSNKALP | |
| | APIEKTISKAKGQPREPQVYTLPPSREEMTK | |
| | NQVSLTCLVKGFYPSDIAVEWESNGQPENN | |
| | YKTTPPVLDSDGSFFLYSKLTVDKSRWQQG | |
| | NVFSCSVMHEALHNHYTQKSLSLSPGK | |

TABLE 2
Mixed chain antibodies of the invention

| Antibody | Heavy chain | Light chain |
|---|---|---|
| RQCov-05/02 | RQCov-05 | RQCov-02 |
| RQCov-05/03 | RQCov-05 | RQCov-03 |
| RQCov-05/04 | RQCov-05 | RQCov-04 |
| RQCov-05/05 | RQCov-05 | RQCov-01/05/06/07* |
| RQCov-07/02 | RQCov-07 | RQCov-02 |
| RQCov-07/03 | RQCov-07 | RQCov-03 |
| RQCov-07/04 | RQCov-07 | RQCov-04 |
| RQCov-07/07 | RQCov-07 | RQCov-01/05/06/07* |
| RQCov-06/02 | RQCov-06 | RQCov-02 |
| RQCov-06/03 | RQCov-06 | RQCov-03 |
| RQCov-06/04 | RQCov-06 | RQCov-04 |
| RQCov-06/06 | RQCov-06 | RQCov-01/05/06/07* |

*RQCov-01, RQCov-05, RQCov-06 and RQCov-07 differ in their heavy chains but share a common light chain.

The antibodies of the invention may be engineered antibodies which have been modified such that they not comprises an N-glycosylation site in the heavy chain variable region and/or the light chain variable region. Preferably, the antibodies of the invention do not comprises an N-glycosylation site in the heavy chain variable region in VHCDR3. The present inventors have surprisingly found that the antibodies of the present invention retain their potency against SARS-COV2 when the glycosylation motif “NRT” in VHCRD3 is removed by substituting the amino acid residue asparagine (N) with a specific alternative amino acid residue.

In a preferred embodiment, the asparagine (N) in the glycosylation motif “NRT” is substituted with an aspartic acid (D) to form the motif “DRT”. Thus, in preferred embodiments, the antibodies of the present invention comprise an asparagine (N) to aspartic acid (D) substitution at position 6 of the VHCDR3 of antibody RQCov-01 (SEQ ID NO: 29), such that the N-glycosylation motif “NRT” is substituted with the motif “DRT” (SEQ ID NO: 3).

In a further preferred embodiment, the asparagine (N) in the glycosylation motif “NRT” is substituted with a Serine(S) to form the motif “SRT”. Thus, in preferred embodiments, the antibodies of the present invention comprise an asparagine (N) to Serine(S) substitution at position 6 of the VHCDR3 of antibody RQCov-01 (SEQ ID NO: 29), such that the N-glycosylation motif “NRT” is substituted with the motif “SRT” (SEQ ID NO: 31).

In a further preferred embodiment, the asparagine (N) in the glycosylation motif “NRT” is substituted with a Glutamine (Q) to form the motif “QRT”. Thus, in preferred embodiments, the antibodies of the present invention comprise an asparagine (N) to Glutamine (Q) substitution at position 6 of the VHCDR3 of antibody RQCov-01 (SEQ ID NO: 29), such that the N-glycosylation motif “NRT” is substituted with the motif “QRT” (SEQ ID NO: 34).

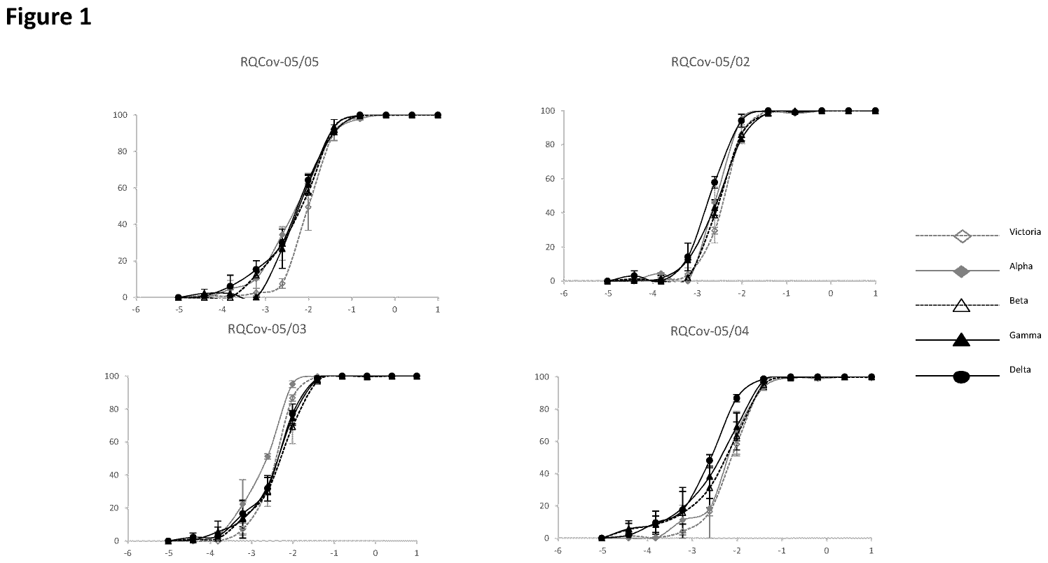

It was surprisingly found that these antibodies bound strongly to SARS-COV-2 and retained the ability to neutralise multiple SARS-COV-2 variants without a loss in potency (see FIGS. 1 to 3 and Example 1).

The term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term also covers any polypeptide or protein having a binding domain which is, or is homologous to, an antibody binding domain. Antibodies may be polyclonal or monoclonal. These can be derived from natural sources, or they may be partly or wholly synthetically produced. “Specifically binds” as used herein means that the antibody binds with a high affinity to the desired antigen and does not significantly bind to, or cross react with, other antigens.

Antibodies are polypeptides that typically contain two identical heavy chains and two identical light chains. In mammals there are two types of light chain, which are called lambda (A) and kappa (κ). Each of the heavy chains and each of the light chains are composed of a variable region and a constant region. The heavy chain variable region is referred to as the VH region and the light chain variable region is referred to as the VL region. For kappa light chains, the VL region can also be referred to as the VK region. Preferably, antibodies according to the invention comprise a Kappa light chain.

Each of the variable regions of the heavy and light chains comprise three complementarity determining regions (CDRs), i.e., CDR1, CDR2 and CDR3. These are named VHCDR1, VHCDR2 and VHCDR3, and VLCDR1, VLCDR2 and VLCDR3, respectively. Antibodies of any immunoglobulin isotype (e.g., IgG, IgE, IgM, IgD and IgA) and their isotypic subclasses; fragments which comprise an antigen binding domain, such as Fab, F(ab′)2, Fv, scFv, dAb, Fd; and diabodies, are also contemplated in the present invention. Preferably, antibodies according to the invention are IgG antibodies.

In preferred embodiment, the antibody of the invention is a full length antibody, i.e., an antibody consisting of two full length heavy chains, each comprising a variable domain (VH) and constant domains (CH1, CH2 and CH3), and two full length light chains, each comprising a variable domain (VL) domain and a constant domain (CL). Preferably the heavy chain is an IgG1 heavy chain and the light chain is a Kappa light chain.

Unless explicitly stated otherwise, any reference to antibody numbering of the variable domains in the present application refers to numbering according to the IMGT numbering system (http://www.imgt.org; Lefranc MP, 1997, J, Immunol. Today, 18, 509) and any reference to antibody numbering of the constant domains in the present application refers to numbering according to the EU index (as in Kabat, E A. et al., 1991, Sequences of proteins of immunological interest. 5th Edition).

In another embodiment, the antibody of the invention may be a fragment of whole antibody, specifically an antigen-binding fragment comprising one or more antigen binding regions. It has been shown that fragments of a whole antibody can perform the function of binding antigens. Examples of binding fragments are the Fab fragment consisting of VL, VH, CL and CH1 domains; the Fd fragment consisting of the VH and CH1 domains; the Fv fragment consisting of the VL and VH domains of a single antibody; the dAb fragment which consists of a VH domain; isolated CDR regions; F(ab′)₂ fragments, a bivalent fragment comprising two linked Fab fragments; single chain Fv molecules (scFv), in which a VH domain and a VL domain are linked by a peptide linker which allows the two domains to associate to form an antigen binding site; bispecific single chain Fv dimers and “diabodies”, multivalent or multi-specific fragments constructed by gene fusion.

Preferably, the antibodies of the invention are monoclonal antibodies. Monoclonal antibodies (mAbs) of the invention may be produced by a variety of techniques, including conventional monoclonal antibody methodology.

Antibodies of the invention may also be multispecific (e.g. bispecific) antibodies. A bispecific antibody is an antibody which can bind to two targets simultaneously, such as two antigens or two epitopes on the same antigen. For example, one binding domain of the antibody may bind to an epitope on the spike protein of SARS-COV-2, and the other binding domain may bind to a different antigen or to a different epitope on the same antigen. In one embodiment, a bispecific antibody of the invention may bind to two separate epitopes on the SARS-COV-2 spike protein. In another embodiment, a bispecific antibody of the invention may bind to the spike protein of SARS-COV-2 and to a different protein. The different protein may be another SARS-COV-2 protein or a non-SARS-COV-2 protein.

The bispecific antibody of the invention can be any suitable format. Examples of bispecific antibody formats include, but are not limited to; (mAb)₂, Fcab, F(mAb′)₂, quadromas, scFv (single chain variable fragments), bsDb (bispecific diabodies), scBsDb (single chain bispecific diabodies), BiTE (bispecific T cell engagers), DART (dual affinity re-targeting antibodies), charge pairs, tandem antibodies, tandem scFv-Fc, Fab-scFv-Fc, Fab-scFv, minibodies, zybodies, DNL-F (ab) 3 (dock-and-lock trivalent Fabs) and bssdAb (bispecific single domain antibodies).

Alternatively, the antigen binding molecules of the invention maybe monovalent for the spike protein of SAR-COV-2, for example a monovalent antibody fragment. A monovalent antigen binding molecule is an antigen binding molecule with only one binding site for an epitope or antigen.

In a further embodiment, antibodies or antigen binding molecules of the invention may also be conjugated to other molecules such as drugs, prodrugs or toxic moieties.

In some embodiments, the antibodies of the invention comprise an Fc region. In one embodiment, an antibody of the invention may comprise a human Fc region, for example an IgA, IgD, IgE, IgG or IgM Fc region. Preferably, the antibody of the invention comprises an IgG Fc region, for example IgG1, IgG2, IgG3 or IgG4. More preferably, the antibody of the invention comprises an IgG1 Fc region.

In some embodiments, the Fc region is a hybrid Fc. A hybrid Fc comprises Fc portions derived from two or more different classes or subclasses of antibody. In some embodiments, the Fc is a IgG1/3 hybrid Fc. In other embodiments, the Fc is a IgG2/4 hybrid Fc.

The antibodies of the invention may comprise modifications in the Fc region. For example, the Fc region of the antibodies of the invention may be modified to improve their stability, increase their half-life and/or to modify their effector function. Suitable modifications are known in the art.

In one embodiment the Fc region may be modified such that it comprises one or more amino acid substitutions relative to a wildtype Fc region of the same isotype. For example, the substitution(s) may remove or enhance binding of the Fc region to an Fc receptor, increase or eliminate an effector function such as antibody-dependent cellular cytotoxicity (ADCC) and/or complement-dependent cytotoxicity (CDC), increase or decrease half-life of the antibody, potentiate immune complex formation, or any combination thereof.

In one embodiment, an antibody of the invention may comprise Fc region modification(s) that alter the half-life of the antibody. In some embodiments, an antibody of the invention may be modified to promote the interaction of the Fc domain with FcRn. In some embodiments, the Fc domain modification(s) are selected from the combinations M252Y/S254T/T256E (‘YTE’), M428L/N434S (‘LS’), S239D/1332E (‘DE’), M252Y/T256D (‘YD’), T256D/T307Q (‘DQ’) T256D/T307W (‘DW’) and/or G236A/A330L/1332E (GAALIE) (numbered according to EU numbering). In a preferred embodiment, an antibody of the invention has one or more modification(s) that alter the serum half-life of the antibody. In a particularly preferred embodiment, the M252Y/S254T/T256E (YTE) mutation may be used to improve the stability and serum half-life of the antibody. Such modifications may be present in addition to modifications which alter Fc effector function.

In some embodiments, the antibody may also be modified to alter the interaction of the antibody with other receptors, such as FcγRI, FcγRIIa, FcγRIIc, FcγRIIIa, FcγRIIB, FcγRIII, and FcαR. Such modifications may increase or decrease the effector functions of the antibody.

In one embodiment, an antibody of the invention comprises a “silenced” Fc region. For example, in one embodiment an antibody of the invention does not display the antibody-dependent cellular cytotoxicity (ADCC) and/or complement-dependent cytotoxicity (CDC) or functions associated with a normal Fc region and/or does not bind to one or more activating Fcγ receptors.

In another embodiment, an antibody of the invention comprises an Fc region with enhanced effector function. For example, in one embodiment an antibody of the invention displays increased ADCC and/or CDC function relative to the ADCC and/or CDC function associated with a normal Fc region.

In one embodiment, an antibody of the invention does not bind to Fc receptors. For example, the antibody does not bind to one or more type of Fc receptor. In one embodiment, an antibody of the invention does not bind FcγR receptors. In one embodiment an antibody of the invention does not bind one or more or all of the activating Fc receptors FcγRI, FcγRIIa, FcγRIIc, FcγRIIIa. In one embodiment, an antibody of the invention does not bind the inhibitory Fc receptor FcγRIIb receptor. In one embodiment, an antibody of the invention does not bind complement. In an alternative embodiment, an antibody of the invention does not bind FcγR, but does bind complement.

In one embodiment, an antibody of the invention binds Fc receptors with enhanced affinity. In one embodiment, an antibody of the invention binds Fcγ receptors with enhanced affinity. In one embodiment, an antibody of the invention binds with enhanced affinity to one of more of the activating Fc receptors FcγRI, FcγRIIa, FcγRIIc, FcγRIIIa. In one embodiment, an antibody of the invention binds with enhanced affinity to the inhibitory Fc receptor FcγRIIb receptor. In one embodiment, an antibody of the invention binds with enhanced affinity to complement. In one embodiment, an antibody of the invention binds with enhanced affinity to FcRn.

In one embodiment, the Fc region of an antibody of the invention does not comprise a CH2 domain. In one embodiment, the Fc does not comprise a CH3 domain. In one embodiment, the Fc comprises additional CH2 and/or CH3 domains.

In some embodiments, cysteine residue(s) can be introduced into the Fc region, thereby allowing interchain disulphide bond formation in this region.

In other embodiments, the one or more substitution(s) may promote heterodimerisation of the antibody heavy chains/and or light chains. For example, where the antibody of the invention is a bispecific antibody, the Fc region of the first heavy chain and the Fc region of the second heavy chain may each comprise one or more substitution(s) which promote selective association of the first heavy chain and the second heavy chain. Suitable strategies for promoting Fc heterodimerisation are known in the art and include steric complementarity (e.g. Knob-in-Hole technology), electrostatic complementarity (e.g. DD-KK substitutions), isotype strand exchange (e.g. SEED) or combinations thereof. The skilled person would appreciate that any suitable heterodimerisation strategy can be applied to bispecific antibodies of the invention.

Alternatively, or in addition, the CH1 region of the first heavy chain and the CL region of the first light chain may each comprise one or more substitution(s) which promote selective association of the first heavy chain with the first light chain and/or the CH1 region of the second heavy chain and the CL region of the second light chain may each comprise one or more substitution(s) which promote selective association of the second heavy chain with the second light chain. Suitable strategies for promoting heavy-chain light-chain heterodimerisation are also known in the art. The skilled person would appreciate that any suitable heterodimerisation strategy can be applied to bispecific antibodies of the invention.

“Identity” as known in the art is the relationship between two or more polypeptide sequences or two or more polynucleotide sequences, as determined by comparing the sequences. Identity, as used herein, may be used interchangeably with “homology” and “similarity”.

References to particular % identities apply equally to % homology and % similarity. The percent identity of two amino acid sequences, or of two nucleic acid sequences, is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100). Generally, references to % identity herein refer to % identity along the entire length of the molecule, unless the context specifies or implies otherwise. The determination of percent identity between two sequences can be accomplished using a mathematical algorithm known to those of skill in the art. Appropriate algorithms include FASTA, BLAST and Gapped BLAST. Software for performing these analyses are publicly available.

In preferred embodiments, the amino acid sequences of the VH, VL, CH1, CH2 and CH3 regions of the antibodies of the invention have at least 70% identity to the amino acid sequences in Table 1. More typically, the VH and VL regions have at least 80%, 85%, 90%, 91%, 92%, 93%, 94%, 95%, 96%, 97%, 98% or at least 99% identity, at the amino acid level, to the amino acid sequences in Table 1.

Accordingly, in one embodiment, an antibody, fragment or variant thereof, is provided comprising a heavy chain variable region comprising a VHCDR1 comprising at least 70%, 75%, 80%, 85%, 90%, 91%, 92%, 93%, 94%, 95%, 96%, 97%, 98%, 99% or 100% identity to the amino acid of SEQ ID NO: 1, a VHCDR2 comprising at least 70%, 75%, 80%, 85%, 90%, 91%, 92%, 93%, 94%, 95%, 96%, 97%, 98%, 99% or 100% identity to the amino acid sequence of SEQ ID NO: 2, a VHCDR3 comprising at least 70%, 75%, 80%, 85%, 90%, 91%, 92%, 93%, 94%, 95%, 96%, 97%, 98%, 99% or 100% identity to the amino acid sequence of SEQ ID NO: 3, SEQ ID NO: 31 or SEQ ID NO: 34; and/or a light chain variable region comprising a VLCDR1 comprising at least 70%, 75%, 80%, 85%, 90%, 91%, 92%, 93%, 94%, 95%, 96%, 97%, 98%, 99% or 100% identity to the amino acid sequence of SEQ ID NO: 6, a VLCDR2 comprising at least 70%, 75%, 80%, 85%, 90%, 91%, 92%, 93%, 94%, 95%, 96%, 97%, 98%, 99% or 100% identity to the amino acid sequence of SEQ ID NO: 7 and a VLCDR3 comprising at least 70%, 75%, 80%, 85%, 90%, 91%, 92%, 93%, 94%, 95%, 96%, 97%, 98%, 99% or 100% identity to the amino acid sequence of SEQ ID NO: 8. In a preferred embodiment, VHCDR3 is 100% identical to the amino acid sequence of SEQ ID NO: 3, SEQ ID NO: 31 or SEQ ID NO: 34.

In one embodiment, the invention provides an antibody that binds to SARS-COV-2, comprising a heavy chain variable region having at least 70%, 75%, 80%, 85%, 90%, 91%, 92%, 93%, 94%, 95%, 96%, 97%, 98% or 99% identity to the amino acid sequence of SEQ ID NO: 4, SEQ ID NO: 32 or SEQ ID NO: 35, and/or a light chain variable region having at least 70%, 75%, 80%, 85%, 90%, 91%, 92%, 93%, 94%, 95%, 96%, 97%, 98% or 99% identity to the amino acid sequence of SEQ ID NO: 9, 14, 19 or 24.

The antibody of the invention is typically a monoclonal antibody. In a preferred embodiment, the antibody is a fully-human monoclonal antibody, in which the human constant region is employed. Preferably, the antibody is a fully-human antibody derived from a recovered SARS-CoV-2 COVID-19 patient.

The antibodies of the invention can be manufactured using any suitable expression system known in the art. For example, Chinese hamster ovary (CHO) cells are suitable for expressing the antibodies of the invention. RQCov-05/03 shows excellent expression and is therefore particularly suitable for use in certain embodiments of the invention.

It is possible to take monoclonal and other antibodies and use techniques of recombinant DNA technology to produce other antibodies or chimeric molecules. Such techniques may involve combining DNA encoding the immunoglobulin variable region, or the complementary determining regions (CDRs), of one antibody with the DNA encoding the constant regions, or the constant regions plus framework regions, of a different antibody. A hybridoma or other cell producing an antibody may be subject to genetic mutation or other changes, which may or may not alter the binding specificity of antibodies produced. In a preferred embodiment, the antibody is a chimeric antibody comprising a light chain variable domain derived from a first antibody clone and a heavy chain variable domain derived from a second antibody clone.

The present invention also extends to variants of the sequences in Table 1 which include one or more amino acid additions, deletions, substitutions or the like. Amino acid substitutions may be made, for example, to improve the stability, manufacturability or functional properties of the molecule.

The skilled person is aware that various amino acids have similar properties. One or more such amino acids can often be substituted by one or more other such amino acids without eliminating a desired activity of that substance. Alternatively, amino acids which do not have a substantial effect on the activity of the polypeptide, or at least which do not eliminate such activity, can be deleted. Substitutions of this nature are often referred to as “conservative” or “semi-conservative” amino acid substitutions.

In one embodiment, the antibodies of the invention comprise one or more amino acid substitutions. In some embodiments, the antibodies of the invention contain one or more “conservative” or “semi-conservative” amino acid substitutions. In some embodiments, the one or more amino acid substitutions are in the CDR region or regions. In other embodiments, the one or more amino acid substitutions are in the framework regions, i.e. in the variable heavy and light chains but not in the CDR region or regions. In other embodiments, the one or more amino acid substitutions may be at any position in the variable heavy and/or variable light regions. In some embodiments, the amino acid substitutions do not adversely affect the binding specificity and/or affinity of the antibody. Accordingly, the variant antibody may have the same or a superior functional profile as the antibody from which it is derived.

Amino acid substitutions or insertions within the scope of the present invention can be made using naturally occurring or non-naturally occurring amino acids, although naturally occurring amino acids may be preferred. For example, the amino acid substitution may comprise a labelled or non-natural amino acid, providing the function of the antibody is not significantly adversely affected.

In some embodiments, the amino acid substitutions all occur in the CDRs of the variable regions. In some embodiments, all the amino acid substitutions occur in the framework regions of the variable regions.

Such antibodies may retain the functional activity (for example EC₅₀, IC₅₀, IC₈₀ and/or K_{D}) of the antibody from which the variant antigen-binding molecule is derived.

Antibodies of the invention may also be modified to improve their potency, to adapt to new SARS-COV-2 variants, or to broaden their ability neutralise multiple SARS-COV-2 variants, for example to identify possible substitutions in the antibody that will compensate for the change in the epitope characteristics.

Modification of antibodies of the invention as described above may be prepared during synthesis of the antibody or by post-production modification, or when the antibody is in recombinant form. Suitable techniques are known in the art, for example site-directed mutagenesis, random mutagenesis, enzymatic cleavage and/or ligation of nucleic acids.

In a further aspect, the invention provides pharmaceutical compositions comprising one or more antibodies of the invention. For example, the pharmaceutical composition may comprise one, two, three or all of the antibodies provided in Table 2.

In one embodiment, the pharmaceutical composition of the invention comprises an antibody according to the invention and one or more of a pharmaceutically acceptable carrier, buffer, excipient, adjuvant, vehicle, diluent or stabiliser or any combination thereof.

Examples of carriers suitable for use with the present invention include water, saline, buffered saline, dextrose, ethanol, liposomes, glycerol, polyethylene glycol and combinations thereof.

The pharmaceutical composition may be in any suitable form or formulated for use by any suitable method of administration. For example, it may be formulated for oral, buccal, sublingual, rectal, nasal, transdermal, vaginal, parenteral, subcutaneous, intramuscular, intravenous or intradermal routes.

The pharmaceutical compositions of the invention may also contain one or more further therapeutically active agents in addition to the molecule or molecules of the present invention. For example, the pharmaceutical compositions of the invention may comprise one or more further antibodies specifically binding to SARS-COV-2. Alternatively, or in addition, the pharmaceutical compositions of the invention may contain one or more of an anti-viral agent, an anti-inflammatory agent, or another therapeutically active agent.

Examples of suitable an anti-viral agents include Remdesivir, Lopinavir, ritonavir, APN01 and Favilavir. Examples of suitable anti-inflammatory agents include corticosteroids (e.g. Dexamethasone) or non-steroidal anti-inflammatory drugs.

In some embodiments, the formulation of the active drug concentrate can comprise a pharmaceutically acceptable tonicity agent, a buffering agent, and a pharmaceutically acceptable surfactant.

It should be understood that in addition to the ingredients particularly mentioned above, the formulations may also include other agents conventional in the art for the type of formulation in question, for example those suitable for oral administration may include flavouring agents.

Also provided are kits comprising antibodies or other compositions of the invention and instructions for use. The kit may further contain one or more additional active agents, such as additional therapeutic or prophylactic agents.

The invention further relates to the use of the antibodies according to the invention for methods of therapy or diagnosis.

In one aspect, the invention relates to methods of treating coronavirus (e.g. SARS-COV-2) infections, or a disease or complication associated with coronavirus infections (e.g. COVID-19 or long COVID).

In one embodiment, the method comprises administering a therapeutically effective amount of an antibody or a pharmaceutical composition of the invention to a subject suffering from a disease or complication associated with coronavirus (e.g. COVID-19 or long COVID).

The antibody or pharmaceutical composition of the invention may be administered by any suitable method. For example, it may be administered subcutaneously, intravenously, intradermally, orally, intranasally, intramuscularly or intracranially. In preferred embodiment, the antibody or pharmaceutical compositions of the invention is administered intravenously or subcutaneously.

In some embodiments, the invention provides a method of reducing the severity of, or preventing the development or progression of, symptoms or complications associated with coronavirus infection (e.g. COVID-19 or long COVID). In other embodiments, the invention provides a method of preventing coronavirus infection from occurring in a subject, for example, a subject who has been, or is at high risk of being, exposed to coronavirus or a subject who is at high risk of experiencing severe symptoms or complications associated with coronavirus infection (e.g. COVID-19 or long COVID).

The invention also relates to an antibody or a pharmaceutical composition according to the invention for use in a method of treating coronavirus (e.g. SARS-COV-2) infections, or a disease or complication associated therewith (e.g. COVID-19 or long COVID).

In another aspect, the invention relates to a method of formulating a composition for treating coronavirus (e.g. SARS-COV-2) infections, or a disease or complication associated therewith (e.g. COVID-19), wherein said method comprises mixing an antibody according to the invention with a pharmaceutically acceptable carrier to prepare said composition.

In another aspect, the invention relates to the use of an antibody or a pharmaceutical composition according to the invention for the manufacture of a medicament for treating coronavirus (e.g. SARS-COV-2) infections or a disease or complication associated therewith (e.g. COVID-19).

The invention also relates to preventing, treating or diagnosing coronavirus infections caused by any SARS-COV-2 variant, as described herein. COVID-19 may be caused by any SARS-CoV-2 variant.

In a preferred embodiment, the invention relates to preventing, treating or diagnosing an infection caused by a SARS-COV-2 variant from lineage hCoV-19/Wuhan/WIV04/2019, SARS-COV-2/human/AUS/VIC01/2020, Alpha (B.1.1.7), Beta (B.1.351), Gamma (P1), Delta (B1.617.2), or Omicron (B.1.1.529). In some embodiments, the invention relates to preventing, treating or diagnosing an infection caused by a SARS-COV-2 variant comprising mutations in the RBD of the spike protein of SARS-COV-2 at one or more of positions 501, 484, 452, 478 and/or 417 relative to hCoV-19/Wuhan/WIV04/2019. In some embodiments, the invention relates to an infection caused by a SARS-COV-2 variant of concern.

For example, the antibody of the invention may be administered before, after or concurrently with another antibody, or binding fragment thereof, of the invention and/or before, after or concurrently with an anti-viral agent or an anti-inflammatory agent. In one embodiment, all the therapeutic agents may be administered together in a single composition. In another embodiment, each component may be administered separately as part of a combination therapy regimen.

In some embodiments, the methods of the invention may lead to a decrease in the viral load of coronavirus (e.g. SARS-COV-2), e.g. by greater than 10%, greater than 20%, greater than 30%, greater than 40%, greater than 50%, greater than 60%, greater than 70%, greater than 80%, greater than 90%, or 100% compared to pre-treatment. Methods of determining viral load are well known in the art, e.g. infection assays.

Further provided is a method of identifying subjects that have a coronavirus infection (e.g. a SARS-COV-2 infection). For example, the methods and uses of the invention may involve identifying the presence of coronavirus (e.g. SARS-COV-2), or a component or fragment thereof, in a sample.

Accordingly, in another aspect, the method comprises identifying the presence of a coronavirus (e.g. SARS-COV-2) or a component or fragment thereof, in a sample from the subject. The sample may be any suitable sample, for example a tissue biopsy, a throat swab, a nasal swab, or a saliva sample.

In a further aspect, the invention provides a method of identifying the presence of a coronavirus (e.g. SARS-COV-2) or a component or fragment thereof in a sample, the method comprising contacting the sample with an antibody of the invention and determining whether the antibody forms an antibody-antigen complex, wherein the formation of the antibody-antigen complex indicates that the sample is positive for coronavirus (e.g. SARS-COV-2).

In embodiments where the invention relates to detecting the presence of coronavirus in a sample, the antibody preferably contains a detectable label, for example a fluorophore, a small molecule, a nanoparticle, a radioisotope, or an enzyme.

The detection may be carried out in vitro, in vivo or in situ. Any suitable method for determining the presence of an antibody-antigen complex may be used. Suitable methods are known in the art. For example, in vitro detection techniques include ELISAs, Western blots, immunoprecipitations, and immunofluorescence. In vivo techniques include labelled antibodies. The detection techniques may provide a qualitative or a quantitative readout.

In a preferred embodiment, the subject is a human subject in need thereof. However, the subject may also be a non-human animal such as a rat, mouse, rabbit, sheep, pig, cow, cat, or dog. The subject may have symptomatic disease. Alternatively, the subject may be asymptomatic or pre-symptomatic.

The invention also encompasses kits for detecting the presence of coronavirus (e.g. SARS-CoV-2), in a sample. For example, the kit may comprise a labelled antibody according to the invention, a means for determining the presence or amount of coronavirus (e.g. SARS-COV-2) in a sample and means for comparing the amount of coronavirus (e.g. SARS-COV-2) in the sample with a standard or control. In some embodiments, the kit is a lateral flow test kit.

The methods of the invention can be used for identifying any SARS-COV-2 variant. In a preferred embodiment, the invention relates to identifying a SARS-COV-2 variant selected from Alpha (B.1.1.7), Beta (B.1.351), Gamma (P1) or Delta (B1.617.2).

The present invention also provides a method of detecting SARS-COV-1 in a sample using an antibody or a pharmaceutical composition according to the invention. For example, the detection of SARS-COV-1 for the diagnosis of SARS-COV-1 infection, or a disease or complication associated with SARS-COV-1.

The antibodies of the invention may be effective in neutralising multiple SARS-COV-2 variants.

A SARS-COV-2 variant is a version of SARS-COV-2 which comprises changes in the genetic sequences of the virus when compared to the reference sequence of hCoV-19/Wuhan/WIV04/2019 (GenBank: MN908947).

In particular, the antibodies of the invention may be effective in neutralising SARS-COV-2 variants which have been designated ‘Variants of Concern’ (VOC) and/or ‘Variants of Interest’ (VOI).

By ‘Variants of Concern and ‘Variants of Interest’, it is meant variants of SARS-COV-2 which are currently designated as such by the World Health Organisation (WHO) according to their current working definitions or any other relevant criteria. Variants currently designated as ‘Variants of Concern’ and ‘Variants of Interest’, are known to the skilled person, and are listed, for example, at https://www.who.int/en/activities/tracking-SARS-COV-2-variants/. Databases documenting the extent of SARS-COV-2 genetic variation can be found at Nextstrain (https://nextstrain.org/), GISAID (https://www.gisaid.org/) and COG-UK (https://www.cogconsortium.uk/).

Designated ‘Variants of Concern’ as of October 2021 are Alpha (B.1.1.7), Beta (B.1.351), Gamma (P1) Delta (B1.617.2) variants. Designated ‘Variants of Interest’ (VOI) as of October 2021 are Lambda (C.37) and Mu (B.1.621) variants.

It would be appreciated by the skilled person that due to the continuous evolution of the SARS-CoV-2 virus and the ongoing research into the impact of these variants, these designations are not fixed, and variants may be added to, removed from, or moved between these designations as deemed necessary, for example, where further changes to the viral genome leads to the emergence of new variants or where new developments in our understanding of the impact of existing variants require a require a reassessment of the risk they pose.

Thus, a previously designated ‘Variant of Interest’ (VOI) or ‘Variant of Concern’ (VOC) which has been conclusively demonstrated to no longer pose a major added risk to global public health compared to other circulating SARS-COV-2 variants, can be reclassified. Conversely, a variant which was not a previously designated Variant of Interest (VOI) or Variant of Concern (VOC) may be designated as such if it is judged by the WHO or relevant public health bodies to meet the relevant criteria as outlined in the working definitions, or if it is otherwise judged to pose sufficient added risk to warrant such designation.

It would also be appreciated that the working definition provided by the World Health Organisation (WHO) for ‘Variant of Interest’ (VOI) or ‘Variant of Concern’ (VOC) may be periodically adjusted to account for e.g. the changing public health situation, the emergence of new variants, or further developments in our understanding of the relative public health risk posed by different variants.

As of October 2021, the term ‘Variant of Interest’ (VOI) is understood to refer to a SARS-COV-2 variant with genetic changes that are predicted or known to affect virus characteristics such as transmissibility, disease severity, immune escape, diagnostic or therapeutic escape and identified to cause significant community transmission or multiple COVID-19 clusters, in multiple countries with increasing relative prevalence alongside increasing number of cases over time, or other apparent epidemiological impacts to suggest an emerging risk to global public health.

As of October 2021, the term ‘Variant of Concern’ (VOC) is understood to refer to a SARS-CoV-2 variant that meets the definition of a VOI (see above) and, through a comparative assessment, has been demonstrated to be associated with one or more of the following changes at a degree of global public health significance:
Increase in transmissibility or detrimental change in COVID-19 epidemiology; or
Increase in virulence or change in clinical disease presentation; or
Decrease in effectiveness of public health and social measures or available diagnostics, vaccines, therapeutics.

The WHO's current working definitions of ‘Variants of Interest’ (VOI) and ‘Variants of Concern’ (VOC) as they relate to SARS-COV2 are known to the skilled person, and are listed, for example, at https://www.who.int/en/activities/tracking-SARS-COV-2-variants/.

It is to be understood that different applications of the disclosed antibody or pharmaceutical composition of the invention may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural references unless the content clearly dictates otherwise. Thus, for example, reference to “an antibody” includes two or more “antibodies”.

All publications, patents and patent applications cited herein, whether supra or infra, are hereby incorporated by reference in their entirety.

### EXAMPLES

### Example 1: Neutralisation of SARS-COV-2

Serially diluted RQCov-05/02, RQCov-05/03, RQCov-05/04, RQCov-05/05, RQCov-06/03, RQCov-07/02, RQCov-07/03, or RQCov-07/07 monoclonal antibody (mAb) was mixed with SARS-COV-2 viruses and incubated for 1 hr at 37° C. The mixtures were then transferred to Vero cell monolayers in duplicate and incubated for a further 2 hrs followed by the addition of 1.5% semi-solid carboxymethyl cellulose (CMC) overlay medium to each well to limit virus diffusion. A focus (infected cell) forming assay was then performed by staining Vero cells with human anti-NP mAb (mAb206) followed by peroxidase-conjugated goat anti-human IgG (A0170; Sigma). Finally, the foci (approximately 100 per well in the absence of antibodies), were visualized by adding TrueBlue Peroxidase Substrate. Virus-infected cell foci were counted on the classic AID EliSpot reader using AID ELISpot software. The percentage of focus reduction was calculated and IC₅₀ and IC₈₀ was determined using the probit program from the SPSS package.

For further details see: Dejnirattisai et. al. (2021). Antibody evasion by the P.1 strain of SARS-CoV-2 Cell 184 (11) 2939-2954 https://doi.org/10.1016/j.cell.2021.03.055.

As shown in FIGS. 1 to 3 and Tables 3 to 8, antibodies RQCov-05/02, RQCov-05/03, RQCov-05/04, RQCov-05/05, RQCov-06/03, RQCov-07/02, RQCov-07/03, and RQCov-07/07 were all highly effective at neutralising all COVID variants tested.

FIG. 1 and Tables 3 and 4 show neutralisation of COVID variants by antibodies RQCov-05/02, RQCov-05/03, RQCov-05/04 and RQCov-05/05.

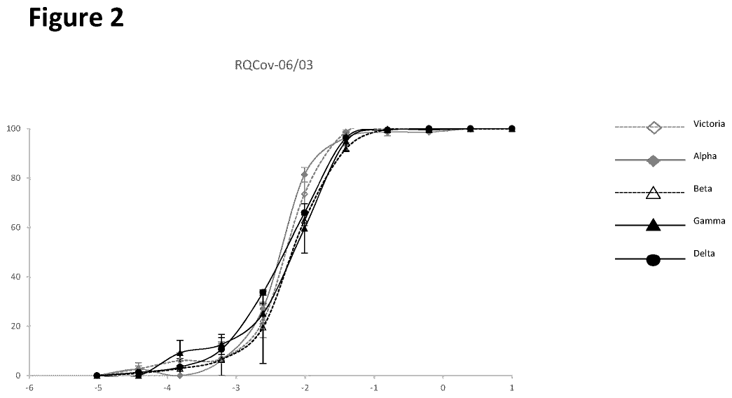

FIG. 2 and Tables 5 and 6 show neutralisation of COVID variants by antibody RQCov-06/03.

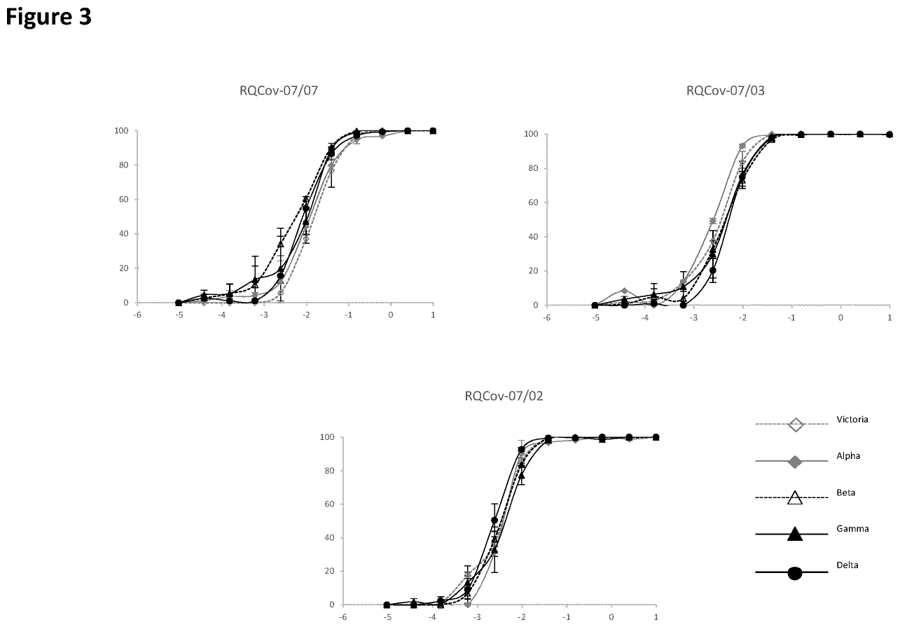

FIG. 3 and Tables 7 and 8 show neutralisation of COVID variants by antibodies RQCov-07/02, RQCov-07/03 and RQCov-07/07.

TABLE 3

| | IC50 (ug/ml) | | | | |
|---|---|---|---|---|---|
| | Victoria | Alpha | Beta | Gamma | Delta |
| RQCov-05/05 | 0.01 ± 0.003 | 0.005 ± 0.002 | 0.005 ± 0.001 | 0.006 ± 0.001 | 0.004 ± 0.001 |
| RQCov-05/02 | 0.004 ± 0 | 0.003 ± 0 | 0.004 ± 0.001 | 0.003 ± 0 | 0.002 ± 0 |
| RQCov-05/03 | 0.003 ± 0.001 | 0.002 ± 0 | 0.004 ± 0.002 | 0.004 ± 0.001 | 0.003 ± 0.001 |
| RQCov-05/04 | 0.007 ± 0.001 | 0.007 ± 0.004 | 0.005 ± 0.003 | 0.003 ± 0.002 | 0.002 ± 0.001 |

TABLE 4

| | IC80 (ug/ml) | | | | |
|---|---|---|---|---|---|
| | Victoria | Alpha | Beta | Gamma | Delta |
| RQCov-05/05 | 0.027 ± 0.004 | 0.02 ± 0.004 | 0.019 ± 0.001 | 0.02 ± 0.007 | 0.019 ± 0 |
| RQCov-05/02 | 0.008 ± 0.001 | 0.007 ± 0.001 | 0.009 ± 0.003 | 0.009 ± 0.001 | 0.006 ± 0.002 |
| RQCov-05/03 | 0.008 ± 0.001 | 0.005 ± 0.001 | 0.014 ± 0.003 | 0.012 ± 0.003 | 0.012 ± 0.005 |
| RQCov-05/04 | 0.022 ± 0.007 | 0.02 ± 0.008 | 0.019 ± 0.008 | 0.014 ± 0.005 | 0.007 ± 0 |

TABLE 5

| | IC50 (ug/ml) | | | | |
|---|---|---|---|---|---|
| | Victoria | Alpha | Beta | Gamma | Delta |
| RQCov-06/03 | 0.004 ± 0.001 | 0.004 ± 0.001 | 0.006 ± 0.002 | 0.005 ± 0.002 | 0.004 ± 0.001 |

TABLE 6

| | IC80 (ug/ml) | | | | |
|---|---|---|---|---|---|
| | Victoria | Alpha | Beta | Gamma | Delta |
| RQCov-06/03 | 0.015 ± 0.004 | 0.015 ± 0.001 | 0.02 ± 0.003 | 0.02 ± 0.006 | 0.015 ± 0.001 |

TABLE 7

| | IC50 (ug/ml) | | | | |
|---|---|---|---|---|---|
| | Victoria | Alpha | Beta | Gamma | Delta |
| RQCov-07/07 | 0.016 ± 0.002 | 0.011 ± 0.001 | 0.005 ± 0.002 | 0.008 ± 0.01 | 0.009 ± 0.001 |
| RQCov-07/02 | 0.003 ± 0 | 0.004 ± 0 | 0.003 ± 0 | 0.004 ± 0 | 0.002 ± 0.001 |
| RQCov-07/03 | 0.003 ± 0 | 0.002 ± 0 | 0.004 ± 0.001 | 0.004 ± 0 | 0.006 ± 0.001 |

TABLE 8

| | IC80 (ug/ml) | | | | |
|---|---|---|---|---|---|
| | Victoria | Alpha | Beta | Gamma | Delta |
| RQCov-07/07 | 0.045 ± 0.015 | 0.056 ± 0.007 | 0.019 ± 0.001 | 0.029 ± 0.008 | 0.034 ± 0.003 |
| RQCov-07/02 | 0.008 ± 0.001 | 0.012 ± 0 | 0.008 ± 0.001 | 0.013 ± 0.004 | 0.006 ± 0.001 |
| RQCov-07/03 | 0.009 ± 0.001 | 0.007 ± 0 | 0.012 ± 0 | 0.013 ± 0.005 | 0.015 ± 0.001 |

1. An antibody capable of binding to the spike protein of the SARS-COV-2 coronavirus, wherein the antibody comprises a heavy chain variable region comprising a VHCDR1 comprising the amino acid sequence of SEQ ID NO 1; a VHCDR2 comprising the amino acid sequence of SEQ ID NO 2; and a VHCDR3 comprising the amino acid sequence of SEQ ID NO 3, 31 or 34; and/or
(a) a light chain variable region comprising a VLCDR1 comprising the amino acid sequence of SEQ ID NO: 6; a VLCDR2 comprising the amino acid sequence of SEQ ID NO: 7; and a VLCDR3 comprising the amino acid sequence of SEQ ID NO: 8;
(b) a light chain variable region comprising a VLCDR1 comprising the amino acid sequence of SEQ ID NO: 11; a VLCDR2 comprising the amino acid sequence of SEQ ID NO: 12; and a VLCDR3 comprising the amino acid sequence of SEQ ID NO: 13;
(c) a light chain variable region comprising a VLCDR1 comprising the amino acid sequence of SEQ ID NO: 16; a VLCDR2 comprising the amino acid sequence of SEQ ID NO: 17; and a VLCDR3 comprising the amino acid sequence of SEQ ID NO: 18; or
(d) a light chain variable region comprising a VLCDR1 comprising the amino acid sequence of SEQ ID NO: 21; a VLCDR2 comprising the amino acid sequence of SEQ ID NO: 22; and a VLCDR3 comprising the amino acid sequence of SEQ ID NO: 23.

2. An antibody according to claim 1 wherein the antibody specifically binds to:
(a) an epitope on the S1 subunit of the spike protein, optionally an epitope on the receptor binding domain (RBD) of the S1 subunit; or
(b) a quaternary epitope that arises through binding higher order structures constituting the trimeric spike protein.

3. An antibody according to claim 1 or 2 wherein the antibody comprises:
(a) a heavy chain variable domain having at least 80% sequence identity to SEQ ID NO: 4, 32 or 35; and
(b) a light chain variable domain having at least 80% sequence identity to SEQ ID NO: 9, 14, 19 or 24.

4. The antibody according to any one of claims 1-3 wherein the antibody further comprises an IgG1 Fc region.

5. The antibody according to claim 4 wherein the Fc region further comprises one or more amino acid substitutions relative to the wildtype IgG1 Fc sequence (SEQ ID NO: 26).

6. The antibody according to claim 5 wherein the Fc region comprises a combination of amino acid substitutions selected from M252Y/S254T/T256E (‘YTE’), M428L/N434S (‘LS’), S239D/1332E (‘DE’), M252Y/T256D (‘YD’), T256D/T307Q (‘DQ’) and/or T256D/T307W (‘DW’) or any combination thereof.

7. An antibody according to any one of claims 1 to 6, wherein the antibody comprises:
(a) two full length IgG1 heavy chains, each having at least 80% sequence identity to SEQ ID NO: 5, 33 or 36; and
(b) two full length kappa light chains, each having at least 80% sequence identity to SEQ ID NO: 10, 15, 20 or 25.

8. The antibody according to any one of claims 1 to 7, wherein the antibody binds to at least two different variants of SARS-COV-2 with a K_{D} value of less than 5 nM, less than 4 nM, less than 3 nM, less than 2 nM, less than 1 nM, less than 0.5 nM, less than 0.4 nM, less than 0.3 nM, less than 0.2 nM or less than 0.1 nM.

9. The antibody according to any one of claims 1 to 8, wherein the antibody has an IC₅₀ value of less than 0.02 g/ml against two or more SARS-COV-2 variants selected from hCoV-19/Wuhan/WIV04/2019 variant, SARS-COV-2/human/AUS/VIC01/2020 variant, Alpha (B.1.1.7) variant, Beta (B.1.351) variant, Gamma (P1) variant, Delta (B1.617.2) variant, Omicron (B.1.1.529) variant, another variant comprising mutations in the RBD of the spike protein of SARS-COV-2 at one or more of positions 501, 484, 452, 478 and/or 417, and/or another Variant of Concern.

10. The antibody according to any one of claims 1 to 9, wherein the antibody has an IC₈₀ value of less than 0.06 μg/ml against two or more SARS-COV-2 variants selected from hCoV-19/Wuhan/WIV04/2019 variant, SARS-COV-2/human/AUS/VIC01/2020 variant, Alpha (B.1.1.7) variant, Beta (B.1.351) variant, Gamma (P1) variant, Delta (B1.617.2) variant, another variant comprising mutations in the RBD of the spike protein of SARS-COV-2 at one or more of positions 501, 484, 452, 478 and/or 417, and/or another Variant of Concern.

11. The antibody according to any one of claims 1 to 10, wherein the antibody comprises:
(a) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 3 and a light chain variable region comprising a VLCDR1 comprising the amino acid sequence of SEQ ID NO: 6, a VLCDR2 comprising the amino acid sequence of SEQ ID NO: 7, and a VLCDR3 comprising the amino acid sequence of SEQ ID NO: 8;
(b) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 31 and a light chain variable region comprising a VLCDR1 comprising the amino acid sequence of SEQ ID NO: 6, a VLCDR2 comprising the amino acid sequence of SEQ ID NO: 7, and a VLCDR3 comprising the amino acid sequence of SEQ ID NO: 8;
(c) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 34 and a light chain variable region comprising a VLCDR1 comprising the amino acid sequence of SEQ ID NO: 6, a VLCDR2 comprising the amino acid sequence of SEQ ID NO: 7, and a VLCDR3 comprising the amino acid sequence of SEQ ID NO: 8;
(d) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 3 and a light chain variable region comprising a VLCDR1 comprising the amino acid sequence of SEQ ID NO: 11, a VLCDR2 comprising the amino acid sequence of SEQ ID NO: 12, and a VLCDR3 comprising the amino acid sequence of SEQ ID NO: 13;
(e) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 31 and a light chain variable region comprising a VLCDR1 comprising the amino acid sequence of SEQ ID NO: 11, a VLCDR2 comprising the amino acid sequence of SEQ ID NO: 12, and a VLCDR3 comprising the amino acid sequence of SEQ ID NO: 13;
(f) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 34 and a light chain variable region comprising a VLCDR1 comprising the amino acid sequence of SEQ ID NO: 11, a VLCDR2 comprising the amino acid sequence of SEQ ID NO: 12, and a VLCDR3 comprising the amino acid sequence of SEQ ID NO: 13;
(g) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 3 and a light chain variable region comprising a VLCDR1 comprising the amino acid sequence of SEQ ID NO: 16, a VLCDR2 comprising the amino acid sequence of SEQ ID NO: 17, and a VLCDR3 comprising the amino acid sequence of SEQ ID NO: 18;
(h) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 31 and a light chain variable region comprising a VLCDR1 comprising the amino acid sequence of SEQ ID NO: 16, a VLCDR2 comprising the amino acid sequence of SEQ ID NO: 17, and a VLCDR3 comprising the amino acid sequence of SEQ ID NO: 18;
(i) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 34 and a light chain variable region comprising a VLCDR1 comprising the amino acid sequence of SEQ ID NO: 16, a VLCDR2 comprising the amino acid sequence of SEQ ID NO: 17, and a VLCDR3 comprising the amino acid sequence of SEQ ID NO: 18;
(j) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 3 and a light chain variable region comprising a VLCDR1 comprising the amino acid sequence of SEQ ID NO: 21, a VLCDR2 comprising the amino acid sequence of SEQ ID NO: 22, and a VLCDR3 comprising the amino acid sequence of SEQ ID NO: 23;
(k) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 31 and a light chain variable region comprising a VLCDR1 comprising the amino acid sequence of SEQ ID NO: 21, a VLCDR2 comprising the amino acid sequence of SEQ ID NO: 22, and a VLCDR3 comprising the amino acid sequence of SEQ ID NO: 23; or
(l) a heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 34 and a light chain variable region comprising a VLCDR1 comprising the amino acid sequence of SEQ ID NO: 21, a VLCDR2 comprising the amino acid sequence of SEQ ID NO: 22, and a VLCDR3 comprising the amino acid sequence of SEQ ID NO: 23.

12. The antibody according to any one of claims 1 to 11, wherein the antibody comprises:
(a) a heavy chain variable domain having at least 80% sequence identity to SEQ ID NO: 4 and a light chain variable domain having at least 80% sequence identity to SEQ ID NO: 9;
(b) a heavy chain variable domain having at least 80% sequence identity to SEQ ID NO: 32 and a light chain variable domain having at least 80% sequence identity to SEQ ID NO: 9;
(c) a heavy chain variable domain having at least 80% sequence identity to SEQ ID NO: 35 and a light chain variable domain having at least 80% sequence identity to SEQ ID NO: 9;
(d) a heavy chain variable domain having at least 80% sequence identity to SEQ ID NO: 4 and a light chain variable domain having at least 80% sequence identity to SEQ ID NO: 14;
(e) a heavy chain variable domain having at least 80% sequence identity to SEQ ID NO: 32 and a light chain variable domain having at least 80% sequence identity to SEQ ID NO: 14;
(f) a heavy chain variable domain having at least 80% sequence identity to SEQ ID NO: 35 and a light chain variable domain having at least 80% sequence identity to SEQ ID NO: 14;
(g) a heavy chain variable domain having at least 80% sequence identity to SEQ ID NO: 4 and a light chain variable domain having at least 80% sequence identity to SEQ ID NO: 19;
(h) a heavy chain variable domain having at least 80% sequence identity to SEQ ID NO: 32 and a light chain variable domain having at least 80% sequence identity to SEQ ID NO: 19;
(i) a heavy chain variable domain having at least 80% sequence identity to SEQ ID NO: 35 and a light chain variable domain having at least 80% sequence identity to SEQ ID NO: 19;
(j) a heavy chain variable domain having at least 80% sequence identity to SEQ ID NO: 4 and a light chain variable domain having at least 80% sequence identity to SEQ ID NO: 24;
(k) a heavy chain variable domain having at least 80% sequence identity to SEQ ID NO: 32 and a light chain variable domain having at least 80% sequence identity to SEQ ID NO: 24; or
(l) a heavy chain variable domain having at least 80% sequence identity to SEQ ID NO: 35 and a light chain variable domain having at least 80% sequence identity to SEQ ID NO: 24.

13. A bispecific antibody comprising:
(a) a first heavy chain comprising a heavy chain variable domain having the sequence of SEQ ID NO: 4, 32 or 35;
(b) a first light chain comprising a light chain variable domain having the sequence of SEQ ID NO: 9, 14, 19, or 24;
(c) a second heavy chain derived from a different antibody clone to the first heavy chain; and
(d) a second light chain, wherein the first heavy chain associates with the first light chain to form a first binding domain specifically binding to an epitope on the spike protein of SARS-CoV-2 or a quaternary epitope that arises through binding higher order structures constituting the trimeric spike protein and the second heavy chain associates with the second light chain to form a second binding domain.

14. The bispecific antibody according to claim 13, wherein:
(a) the second light chain is the same as the first light chain; or
(b) the second light chain is derived from the same antibody clone as the second heavy chain.

15. A polynucleotide encoding the antibody or bispecific antibody according to any one of claims 1-14.

16. A vector comprising the polynucleotide according to claim 15.

17. A host cell comprising the polynucleotide according to claim 15 or the vector according to claim 16.

18. A pharmaceutical composition comprising an antibody or bispecific antibody according to any one of claims 1-14, a polynucleotide according to claim 15, or a vector according to claim 16 and at least one pharmaceutically acceptable diluent or carrier.

19. A pharmaceutical composition comprising two or more antibodies according to any one of claims 1-14, two or more polynucleotides according to claim 15, or two or more vectors according to claim 16 and at least one pharmaceutically acceptable diluent or carrier.

20. The pharmaceutical composition according to claim 18 or 19, wherein the composition further comprises one or more additional antibodies capable of binding to SARS-COV-2.

21. The antibody or bispecific antibody according to any one of claims 1-14, the polynucleotide according to claim 15, the vector according to claim 16, the host cell according to claim 17, or the pharmaceutical composition according to any one of claims 18 to 20 for use as a medicament.

22. The antibody or bispecific antibody according to any one of claims 1-14, the polynucleotide according to claim 15, the vector according to claim 16, the host cell according to claim 17, or the pharmaceutical composition according to any one of claims 18 to 20 for use in a method of treating or preventing a disease or a complication associated with coronavirus infection, optionally wherein the coronavirus is SARS-COV-2.

23. A method for treating a subject having a disease or a complication associated with coronavirus infection, the method comprising administering a therapeutically effective amount of the antibody or bispecific antibody according to any one of claims 1-14, the polynucleotide according to claim 15, the vector according to claim 16, the host cell according to claim 17, or the pharmaceutical composition according to any one of claims 18 to 20 to said subject.

24. The antibody or bispecific antibody according to any one of claims 1-14, the polynucleotide according to claim 15, the vector according to claim 16, the host cell according to claim 17, or the pharmaceutical composition according to any one of claims 18 to 20 for use in the manufacture of a medicament for treating or preventing a disease or a complication associated with coronavirus infection, optionally wherein the disease is COVID-19 or long COVID.

25. A method for producing an antibody that is capable of binding to the spike protein of coronavirus SARS-COV-2, comprising culturing the host cell of the invention and isolating the antibody from said culture.

26. A method of identifying the presence of a coronavirus or a component or fragment thereof in a sample, the method comprising:
(a) contacting the sample with an antibody according to any one of claims 1-12; and
(b) determining whether the antibody forms an antibody-antigen complex;
wherein the formation of the antibody-antigen complex indicates that the sample is positive for coronavirus, optionally wherein the coronavirus is SARS-COV-2.
